(19) United States
(12) Patent Application Publication
(10) Pub. No.: US 2008/0119763 A1
(43) Pub. Date: May 22, 2008

(54) Acquisition processing and reporting physical exercise data

(76) Inventor: Jay Wiener, San Jose, CA (US)

Correspondence Address: Walt Froloff, 273D Searidge Rd, Aptos, CA, 95003, US

(21) Appl. No.: 11/602,912

(22) Filed: Nov. 21, 2006

(51) Int. Cl. A61B 5/22

(52) U.S. Cl. 600/587

(57) ABSTRACT

The present invention discloses a system and method for acquiring, processing and reporting personal exercise data and/or concurrent with other physiological data. on selected muscle or muscle groups by measuring vector force from at least one muscle or muscle group acting on physical exercise equipment whereby applied vector force on an exercise equipment element sends force vector and displacement vector data for determining physiological resistive force, work and performance by a muscle or muscle group. The invention can be adapted to various existing exercise equipment including but not limited to elastic cable, stretching band, weight bearing cable, flexure member, spring, bar, rigid structure, non-stretching cloth, composites, elastics, latex, hypoallergenic elastic, flat sheets, tubes, slates, pipes and fiber

### BACKGROUND

### Field of the Invention

The present invention generally relates to acquiring, processing, storing and reporting data from sensors used with physical exercise equipment and components for human performance metrics. More specifically, the physical exercise measured can be general, therapeutic, isometric or isotonic, and application dependent, with small attachments as necessary for use with existing exercise systems. The data acquisition and management system receives data from a plurality of sources for distribution in fulfillment of a plurality of physical exercise application needs.

The general area of physical exercise data processing and management has grown at a phenomenal rate, as more is learned about the human mind-body and as technology delivers more convenient ways and means of tapping in and processing.

The area of physical exercise contains a large diversity of products. In addition, some systems provide feedback to a user of a weight stack machine having a stack of weight plates for lifting one or more of plates from a stack during lifts. Some of these systems use load cells for determining the weight of the weight plates prior to lift and for determining the weight of weight plates remaining on the stack after the user has lifted the plates. These systems comprise electronic detection means coupled to load cells for computing difference data describing the weight of the plates lifted from stacks, interface means for transmitting data from the electronic detection means to a storage. These systems may also provide means for evaluating the height of lifted weight plates or the distance that the weight stack is pulled.

One problem which arises from use of load cells is that the weight of the stack is not equal to the force used to exercise the stack. Hence the work and force are inaccurately calculated. Other challenges are that the force and work are calculated based on the weight of the weight stack and the work done on the weight stack, and not the work done by the user in exerting a force on that weight. The weight's mass provides only part of the resistance through which a user applies force and work. The work can also be done without a mass moving, strain work. Work can be done by accelerating the mass, not taken account by a straight weight−height calculation. The work done on the weight machine is not the desired quantity. What is needed is the force and work done by the muscle and on the muscle, which is not the same as the work done on an exercise object or weight stack.

Moreover, work done by the user is determined by the dot product of the force vector with the displacement from position vector, to take into account when the displacement is not parallel to the force vector along the force displacement locus. Most systems rely on a simple straight Force times Distance calculations which may yield the work done on the object in the simplest arrangements, without regard to the displacement locus, in determining the work done by the muscle. These systems are of little value to rehabilitation of targeted muscle tissue since the work results calculated bare little relationship to the force applied and work done by the muscle targeted. Determining the weight of the stack lifted or the stack left is not even sufficient to adequately determine force, work, power done on the weights from a purely physics standpoint, since the force used to lift the weights is not equal to the weight, but a function of the acceleration of the mass which is related to the weight. Moreover, the physics work is also a function of geometry, distance, rate of pull or stretch, and other factors. Furthermore, to determine physical force, physical work and power performed by a muscle or muscle group, more than the weight or lift height needs to be included in the calculation. The what and how a muscle is contracted/relaxed and the position that a user takes in the lifting pulling pushing or dragging, the motion that the moving limb makes affects the geometry and therefore the calculation of the physical force, work, power done by a particular muscle or muscle group. What are needed are systems which accurately determine the force, work and power not done in the ideal circumstance on the exercise equipment from the purely physics standpoint, but the force, torque, work and power that the users muscle actually exerts.

Although weight lifting is the traditional method of exercise of a particular muscle, many other methods are used today and by many means. For example, isometric exercises work muscle against other muscle(s) or muscle against non-moving objects, instead of gravity, while isotonic exercise may require even resistance or stretching elastic or flexing composite fiber to maintain a specified resistance in accommodating muscle work. What is needed are systems that have the flexibility to integrate with many other exercises, accommodating customized workouts exercise systems, exercise methods and exercise devices, which can be programmed and measured for physical performance, trends, comparisons, and reports.

In contemporary control applications, weighing systems are used in both static and dynamic applications. Some systems are technologically advanced, interfacing with computers for database integration and using micro-processor based techniques to proportion material inputs and feed rates. To send weight information to computers, signal conditioners are utilized to permit direct communication from a load cell via conversion of the load cell's analog signal to a digital signal. These techniques as applied in the physical exercise arena, has yet to capture the work done by an individual, because the weight of an object is not the force, torque, work or power applied in exercising the muscle. What is needed are systems which can accurately determine the force, work and power acted on by specific muscle or muscle groups in a particular exercise.

Some systems offer physiological parameter monitoring and bio-feedback apparatus and teach a bio-feedback to support the collection of a number of physiological parameter values of a subject being monitored. The physiological parameter values collected are processed to determine and present to the subject a continuously updated succession of presentation states, including multimedia modes, in order to attempt to enhance the bio-feedback capability of the system. These system may include a digitizing camera arranged to continually capture an image of the subject. The presentation states, which include the captured image of the subject, are accented by color accents including curved bands of color coextensive with and juxtaposed to the outline of the image of the subject. The color of the bands may be appropriately altered in a predefined manner as determined by changes in the physiological parameter values collected and processed. These real time presentation of visual data are for general awareness of strength exhibited during an exercise and for physical component state comparison, not for the purposes of self-altering progress monitoring and reporting. What are needed are systems where subject images are not required, where image processing and not the rehabilitating process or need, where presentation need not be in real-time only, whereas reporting on demand for comparisons and reminders requires storage and management and other functions not associated with real-time feedback, which during and after the exercise brings some distinctions as to objectives and functions required. There are some physical therapies where digitizing camera are arranged to continually capture an image of the subject but leave out display of limbs of the subject or alignment of the subject. The limbs and alignment of the limbs as they progress through the exercise are vital elements in calculations for physical work and rehabilitation. What are needed are systems for tracking these elements so that accurate exercise figures be calculated. Some such image processing systems require a computing means having the digitizing camera operatively coupled to captured image of the subject and further operatively coupled to the sensors to enable collection of physiological parameter values determined by the sensors that compose a physiological state. These omit perceptions and calculations on the underlying elements of physiological states, such as muscle and only during the concentric and eccentric phases of contraction, which are not visible by digitized camera images. Muscle contraction is internal and outside the scope of visual image presentation. Processing images for presentation feedback purposes misses much of the true rehabilitation process. While there may be a place for visual presentation, what are needed are systems whereby a digitized visual image of the subject can be used to extract the underlying physics for the calculation of the physical exercise, than translate that to the physiological or actual work exercise of the muscle.

Some vendors offer wireless medical diagnosis and monitoring equipment, which use wireless electrodes attached to the surface of the skin of the patient. The electrodes comprise a digital transmitting and receiving unit with antenna and micro-sensors. The electrodes can be used, among other things, for detecting EEG- and EKG-signals, as well as for monitoring body-breathing movements, the temperature, perspiration, etc. These may contain an electrode comprising all functions in a semiconductor chip which, as an integrated circuit, equipped with the respective sensor, sensor control, frequency generation, transmitting and receiving units, as well as with a transmission control unit. The antenna is arranged in this connection in the flexible electrode covering or directly in the chip.

However, these systems are restricted to electrodes attached to a patient or a wireless connection with evaluator station. Some of these physical exercises require sensor attachment to physical components and equipment, which are outside the patient's physical contact, and external to the patient sensor locations. What are needed are sensors and systems that are flexible, not limited to attached electrodes but that have connectivity from base or central locations to users.

Some wireless internet bio-telemetry monitoring for monitoring patient variables in a wireless mode via patient worn monitoring device connected to a variety of bio-sensors with at least one microphone for voice communications. The pertinent worn device connects to a wireless network and thence to the Internet for transmitting voice and data to a health care provider. The benefit is that the health care provider communicates with the patient worn device via the internet and the wireless network to send instructions to the patient worn monitoring unit and to communicate via voice with the patent. The health care provider can also flexibly reconfigure the patent worn monitoring device to change collection parameters for the bio-sensors worn by the patient. When an alarm limit is exceeded and detected by the bio-sensors, it is transmitted to the health care provider over the wireless network and then over the internet thereby allowing full mobility to the patient wearing the device.

However, the purpose of these systems are for keeping track of elderly and partially disabled for health care providers and these systems are narrowly tailored for those purposes. There are two way voice communication for physical care and well-being which is required by the monitoring. What is needed are systems where exercise data for particular rehabilitation can be acquired, processed, evaluated, monitored and presented in a variety of formats for distribution to various health organizations and or personal use.

Some systems offer a computer program product to produce or direct movements in synergic timed correlation with physiological activity. The synergic programs module causes generation of at least one signal, stimulus, or force, where the movements are performed in response to at least one signal, stimulus, or force, where each of the signals, stimulus, or force is determined so as to reduce meaning and/or emotional content to the subject. Each signal and stimulus is from a pool that comprises signals and stimuli that are understandable or recognizable by the subject, and where timing of the movements are based on at least a primary correlation factor and a secondary correlation factor. The primary correlation factor is determined so that the movements are synchronized with referential points of an intrinsically variable cyclical physiological activity.

What are needed are systems measuring the physical activity expended without a priory stimulus, but from intent to exercise a particular muscle based on knowledge and instruction. What are needed are direction of movements in a temporally varying fashion but in regard and direction to rehabilitation plans and programs, not performed in response to a signal determined so as to reduce meaning and/or emotional content of a subject. Some subjects exercise from emotions of pleasure and some from emotions of pain. The emotional signals are not necessarily causal to exercise and systems are needed which allow flexibility in rehabilitation outside the scope of emotive theory on exercise. Although the emotional well being is a valid goal throughout the rehabilitation process, what are needed are directed movements not based on correlation factors determined so that movements are synchronized with referential points of an intrinsically variable cyclical physiological activity, but in compliance with extrinsically dictated individually created or adopted programs that correctly calculate and monitor re-habilitation progress and status.

Some methods provide apparatus for rehabilitation of neuromotor disorders. A system for individually exercising one or more parameters of hand movement such as range, speed, fractionation and strength in a virtual reality environment and for providing performance-based interaction with the user to increase user motivation while exercising. Some of these are claimed for rehabilitation of neuromotor disorders, such as a stroke. A first input device senses position of digits of the hand of the user while the user is performing an exercise by interacting with a virtual image. A second input device provides force feedback to the user and measures position of the digits of the hand while the user is performing an exercise by interacting with a virtual image. The virtual images are updated based on targets determined for the user's performance in order to provide harder or easier exercises.

These use sensing means adapted for sensing position of one or more digits of a hand sensor data; with force feedback means adapted for applying force feedback to one or more digits and for measuring position of a tip of each of one or more digits in relation to a palm of the hand to provide second sensor data. This systems do not use force feedback of any kind, much less to specific digits of a subjects hand or for measuring position of finger tips. These also use a virtual reality simulation means for determining a virtual image of virtual objects movable by user to virtually simulate an exercise to be performed by the user. The use of virtual reality is not for everyone, and is limited to a very narrow range of physical rehabilitation or even exercise.

Others use signal acquiring apparatus, database, database system, signal presenting systems, and signal acquiring, storing, and presenting data, storing audiovisual signals and bio-signals as experience information. A signal acquirer/encoder collects the audiovisual information and bio-information regarding a user through sensors attached to the user, integrates and encodes these signals, and stores the integrated and encoded signals. Processed data is selected for effective information from the integrated signals on the basis of a comprehensive judgment, and stores the effective information. The storage is connected to a database that includes a personal database allotted to each user. The user stores enciphered integrated signals in the database allotted to the user. The database is connected to a public database to automatically access associated information. The database and an integrated signal presenter are connected, enabling the user to have a re-experience by means of the integrated signal presenter. These system have sensors which must include at least one biometric sensor to sense bio-information of the person and at least one audio/video sensor to sense audio/video perceivable by the person in the person's environment. The person does this so that the audio/video signals of the audio/video perceived by the subject correlated with a change in bio-information, enable a subsequent re-experience when the audio/video signals are reproduced in the person's presence. What are needed are systems that are simpler to understand and believe in, without requirements for audio/video for the purposes of a re-experience, but with real muscle and muscle group work-force-performance measurements.

A wearable data input interface device, offers a method for entering data into a computer device and provides a wearable device that is attached to a hand. The device has extensions disposed below metacarpophalangeal joints and first bone segments of the fingers. The extensions have sensors in operative engagement with sensor channels. The first bone segments may be moved relative to second bone segments at the metacarpophalangeal joints to bend at least one of the sensor channels. One of the sensors sensing the bending of one the sensor channels send an activation signal to a computer device in operative engagement with the device.

Although this devices senses the movement of the bone segment bending the sensor channel and sends an activation signal to a computer device in operative engagement with the device, this system does not sense the muscles nor does it process muscle data. The purpose of that invention is sensor measurements on bones and joints, not position vector changes, force vectors, displacement vectors, or acceleration vectors.

Another system processes pain signals, and applies methods, apparatuses and systems relating to the objective measurement of the subjective perception of subjective pain in a subject, as measured by electrodes. The electrodes measure electrical activity at locations on a subject to generate at least two sets of electrical activity measurements. The system further comprises processing the electrical activity measurements into at least two normalized signals, and comparing the at least two normalized signals to each other to identify the presence of pain in the subject. These are very intrusive systems and what is needed are less intrusive ways to grow or rehabilitate muscle tissue.

Some systems claim medical protocol management, for treating orthopedic injuries by presenting a set of treatment protocols; approving a treatment protocol from among the presented set of treatment protocols; capturing information identifying the approved treatment protocol from among the set of presented treatment protocols; and generating information from the captured information into a form compatible with a handheld computer adapted for connection to an orthopedic sensor system. The generated information includes parameters of the identified approved treatment protocol. This process may also include the steps of basing the presented set of treatment protocols upon a database of historic patients, orthopedic injuries, treatment protocols and outcomes, and retaining information about the current patient, the patient's injury, treatment protocol and outcome. Orthopedics is the practice of treating musculoskeletal disorders and these systems provide automated systems for musculoskeletal treatment protocols. These are accessed from a database of standardized orthopedic treatment protocols for treatment of bones joints tendons. What is needed are systems which accurately measure muscle strength, growth and rehabilitation.

Moreover, because these systems must treat a complex combination of body parts, they are limited to transducer equipped personal orthopedic restraining devices, PORD, or alternatively, a transducer equipped belt. The sensors are located on the subject, which are cumbersome to install and annoyance to wear. What is needed is quick, simple and easy sensor placement on equipment. Furthermore, the PORD restrains or restricts motion of the patient's appendage for rehabilitation to motions consistent with a treatment protocol. Such restrictions inhibit users from finding their own limits in muscle movement and therefore growth. Other limitations preclude these systems from allowing flexibility with much of the equipment available for exercise of muscle.

What are needed are systems which do not necessitate professional supervision but attempt to automate the process to the extent that the patient can self service, providing the data, statistics and monitoring to organizations that may have diverse requirements, outside of the treatment itself, such as insurance, personal goals, rehab requirements regimes, etc. What is needed are systems that have the flexibility to accommodate these exercise regimes, goals, and needs from various equipment, exercise methods and exercise devices.

Cables that are attached to one or more sensors (such as load cells) can be either elastic or inelastic. Systems that have components to work with most commercially available elastic cables and inelastic cables with or without handles designed for exercise are needed.

Elastic Cables, also called Stretching Bands, are commonly used for resistive exercise, both for rehabilitative therapy and for general fitness. Most stretching bands are made from latex; some are made from hypoallergenic elastic materials. Stretching bands come in two main shapes: flat sheets and cables, sometimes called “pipes”, “tubes”, or “tubing”. The amount of resistance generated depends on the thickness of a cross-section of the material: the thicker the material, the more resistance required to stretch it a pre-determined distance. Flat stretching bands are typically sold in eight different colors; different colors indicate different thickness of material. What are needed are exercise systems which accommodate these color standards. What are needed are methods to account for the diversity in material stretch properties and their changes in time.

Force exerted against inelastic materials—ropes, steel cables, webbing, etc—so that a user can measure strength during an isometric or isotonic contraction at selected achievable ranges of motion, and at selected achievable angle to the body and/or floor, can also be measured on exercise equipment such as BowFlex™ products and other devices that use flexible materials to generate resistance, Nautilus™ equipment, Universal™ Weight machines, and other products that employ weights that slide up and down pulleys, tracks, bars, etc and other exercise equipment that uses gravity, friction, or mechanical devices, contrivances, etc. to generate resistance. These products do not universally acquire exercise data across other popular systems. What is needed are systems that are cross compatible, taking full advantage of existing exercise system and yet are sufficiently intelligent to understand the exercise physics for translating to analogous physiological force, work and power of human subjects, process, manage and display those data and more.

### SUMMARY

The present invention discloses a system for acquiring, processing and reporting personal exercise data comprising: a transducer element registering an applied vector force from at least one muscle or muscle group acting on physical exercise equipment, a sensing device converting vector force to electronic signal, a data receiving element receiving the sensed signal, an application program comprising a computing device processing the input from data receiving element, and an application program processing vector force and data received from personal exercise, whereby applied vector force on an exercise equipment element sends force vector and displacement vector data for determining resistive force done by a muscle or muscle group.

The invention further comprising determining work or power done by a muscle or muscle group. This can be done through various existing equipment with invention embodiments using personal exercise elements from a set of exercise elements consisting essentially from elastic cable, stretching band, weight bearing cable, flexure member, spring, bar, rigid structure, non-stretching cloth, composites, elastics, latex, hypoallergenic elastic, flat sheets, tubes, slates, pipes and fiber.

### BRIEF DESCRIPTION OF DRAWINGS

FIG. 1 is a force body diagram illustrating an exercise as a sequence of force displacements according to an embodiment of the present invention.

FIG. 2 shows a 3D Acceleration vector and 1D Force vector load cell as an element of an exercise data system in accordance with an embodiment of the invention.

FIG. 3. illustrates a simple isometric exercise with accompanying force and strain energy model for data acquisition in accordance with an embodiment of the invention.

FIG. 4 shows front and side view for a load cell for 2D force vector in accordance with an exercise data system embodiment of the invention

FIG. 5 shows front and top view for a shoulder abduction exercise with force vector and displacement for the purposes of determining force and work done on muscle from the elastic stretch bands exercise in accordance with an embodiment of the invention.

FIG. 6 shows an implemented exercise data system in accordance with an embodiment of the invention.

FIG. 7 shows stretching bands and components of an isotonic exercise data system in accordance with an embodiment of the invention.

FIG. 8 shows a simple calibration and initialization for an exercise using an exercise data system in accordance with an embodiment of the invention.

FIG. 9 shows a high level flow chart for exercise data acquisition and initialization process for an exercise data system in accordance with an embodiment of the invention.

FIG. 10 shows a high level flow diagram for exercise data processing in accordance with an embodiment of the invention.

FIG. 11 illustrates an exemplar display of exercise data in accordance with an embodiment of the invention.

FIG. 12 illustrates smoothness analysis in accordance with an embodiment of the invention.

FIG. 13 illustrates exemplar exercise data identification of abnormalities from a smoothness analysis in accordance with an embodiment of the invention

FIG. 14 shows a table of exemplar exercise data used in consistency analysis in accordance with an embodiment of the invention

FIG. 15 shows a table of exemplar exercise data used in endurance trend analysis in accordance with an embodiment of the invention

FIG. 16 illustrates exemplar exercise data graphs used in time differential smoothness analysis in accordance with an embodiment of the invention.

FIG. 17 illustrates exemplar exercise data graphs used in comparison and trend analysis in accordance with an embodiment of the invention

### DETAILED DESCRIPTION

Specific embodiments of the invention will now be described in detail with reference to the accompanying figures.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

### OBJECTS AND ADVANTAGES

The present invention is a system and method of acquiring physical exercise data and processing that data to correctly calculate the physical forces, torques, work and power exerted by individual users of such a system. The data is processed, stored, and displayed in real-time and reported in a variety of report formats in accordance with user requirements and needs.

Accordingly, it is an object of the present invention to provide an easy to use method and apparatus for rehabilitation of a targeted muscle or muscle group

It is another object of the present invention to provide a method and apparatus for determining and tracking muscle building progress, trends, weaknesses and or tremors.

Embodiments of the invention collect, store and process information for patients and general fitness users while they exercise freely, isometrically or isotonically, using elastic materials or inelastic materials providing exercise resistance.

Other objects of exercise data processing include:

1. Diagnosis of muscle injury, weakness, or other problems

2. Determine precise location of pain

3. To define benchmark levels of strength.

4. To track improvement or decline of strength, endurance, and range of motion

5. Predict future improvement, decline, or return to normal functioning.

6. Automatically suggest changes to exercise routines

7. Allow therapists, trainers, etc., to evaluate exercise programs

8. Determine the location and extent of tremors

An embodiment of the invention measures and records the amount of force applied to an elastic or cable (stretching band) or inelastic cable or exercise machine while a user is exercising generally, isotonically or isometrically. This measurement can be done while a user exerts a force using specific muscle(s) or muscle group(s) and while the subject is progressing through an exercise or routine. In order to accurately calculate the force, work and performance of the muscle, actual forces as exerted by the muscles will require knowledge of the geometry or position from which the manipulation is done, as well as the type of equipment used, so that the correct models can be used in determining the actual required parameters. Together, this information can give a complete description of force, work and power exerted by specific targeted major muscle groups, especially those involving the limbs and joints.

Exercise is a continuous analog function and data can be continuous analog in nature depending on the sensors, sensor types, conditioners, or digitized signal in the data stream occurring upstream of the processor action on the data. Moreover, measurements can be digital, with selected scan rates and these methods are known to those skilled in the art. Data is taken as many times per second as is necessary and appropriate, appropriate in order to track an entire exercise movement from beginning to end and all that in the interim at a granularity necessary to obtain the minimum number of data points for projecting a smooth locus for accurately calculating the physical exercise parameters. In their intensity and desire to grow stronger faster, user's often “cheat” in exercise performance. Thus initial acceleration force used by one part of the muscle will require less force by another part and perhaps weaker part of the muscle. Thus the force as well as acceleration vector as a function of time must be known for some exercises, to establish the direction and locus of the exercise sweep. In some instances, an analog signal must be digitized in order to process the forces, moments acting in specific positions over an interval of time.

The geometry, force and torque calculations may involve some initialization, so that the muscles targeted for exercise can be properly identified, assigned, and physical forces, work and progress properly sensed, acquired and processed in accordance with known geometrical parameters.

Another embodiment of the invention acquires raw exercise data, applying physics principles of force, work and power as well as numerical algorithms to translate the sensed data into appropriate force, work and power from the physiological movement and work and in order to create a variety of reports. This is selectively done for individual workouts, cumulative correlations from historical data from selected previous stored exercise data, to show progress or decline, and offering predictions as to when a patient can return to normal functioning (Maximum Medical Improvement.) These Predictive reports provide athletic trainers with the necessary information to bring athletes to peak performance before athletic events and/or reports can help guide patients and therapists as to the optimum time to return to work. Some embodiments will complete forms that users and their authorized agents (doctors, therapists, trainers, insurance companies, etc.) can access to obtain reports on demand. Because the data is direct performance results on affected muscle, causally related to the exercise(s), the data can be used to pinpoint areas of injury, weakness, or pain.

An aspect of the invention captures exercise data by using sensors to monitor exercise equipment and equipment components rather than of using sensors to monitor the user without the attachment of sensors to the user.

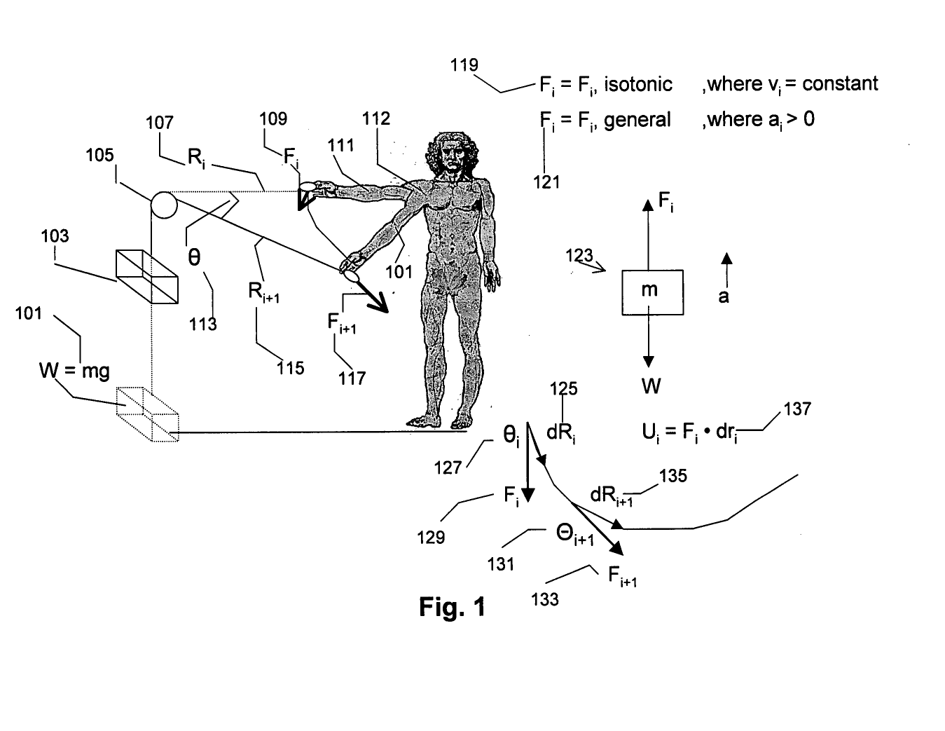

FIG. 1 is a force body diagram illustrating an exercise as a sequence of force displacements along a locus according to an embodiment of the present invention. The shoulder muscle group 112 through extended arm 111 motion applies a force 109 moving an exercise machine cable end to a new arm position 101. The cable is loaded with a weight 101 which is pulled upward to a new position 103 over a pulley 105, changing the position vector R 107 along an arc of angle Θ theta 113 to a new position R 115. From a stationary position, a general Force F 121 required to move the weight 101 must have some acceleration to overcome the mass 101 inertia, shown in 123, is the

F=ma−W,

where F is force, m is mass, a is acceleration of the mass and W is weight of the mass opposing motion. Hence the invention will include steps to calibrate or ascertain the weight and measure acceleration vectors, or acting force vector, as well as associated geometrical parameters defining the displacement locus.

The force F 129 is shown to be action tangent to the displacement dR 125 by theta angle Θ 127. As the arm 111 motion progresses in the exercise, the displacement dR 125 135 and the force F 129 133 must be known, and indeed are scanned and digitized by another aspect of the invention, to determine the work U 137, work being defined is the dot product of the force and displacement vectors. Thus the work in some embodiments is calculated as the component force acting in the direction of displacement multiplied by the displacement. Hence an embodiment of the invention uses the acceleration vector numerically integrated twice to provide displacement locus so that the dot product representing the work can be calculated Thus, a method of determining the resistance with associated exercise motion can be made using acceleration vector and displacement of a point in 3D space with initial parameters or using defaults, in calculating resistance of exercise extension from one position to another position.

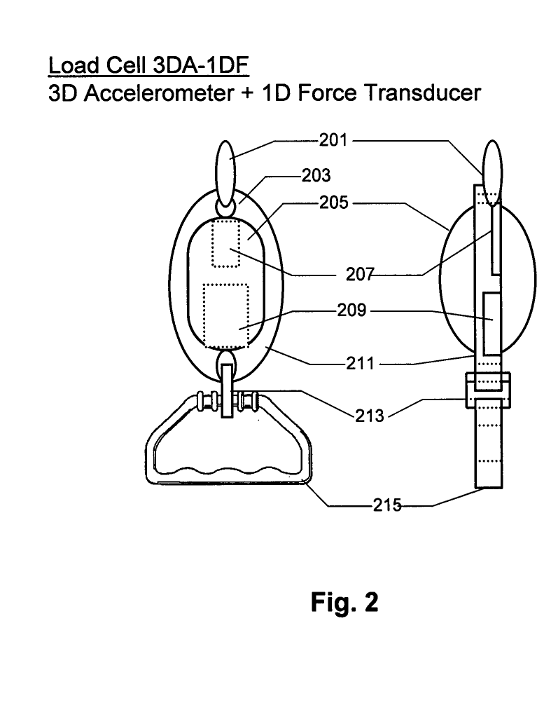

FIG. 2 shows a 3D Acceleration vector and 1D Force vector load cell as an element of an exercise data system in accordance with an embodiment of the invention.

Some load cells are classified as force transducers. This device converts force into an electrical signal. There are many types of load cells, but the precise positioning of the gage, the mounting procedure, and the materials used all have a measurable effect on overall performance of the load cell.

A pod 205 casing houses the load cell 207 and circuitry 209, providing a rigid coupling element 211 with openings 203 for coupling devices 201 213, connecting a handle or gripping 215 element on one and a resisting element or resistance transferring element on the other end. The 3DA-1DF pods detect force along a single exercise axis and associated acceleration vector in three dimensions during an exercise and transmit raw data to the computer for processing. Each pod 205 contains a load cell 207 and circuitry 209 where conversion and processing can be done, and signal outputs or data streams of the measured parameters can be transmitted for further processing, storage or display. In some embodiments, the circuitry is used to convert the data stream from the load cell 207 from analog into digital format. Pods 205 may optionally be communicatively coupled with “WiFi”, Blue Tooth, 802.11B, or similar means of wirelessly transmitting standards and formats. The pod 205 output force can be taken from a handle 215 via a one, two or three dimensional coupler 213. The other pod is further coupled to a cable, exercise resistance element, junction box further integrating load cell signals, instrumentation such as indicators, signal conditioners and the like, as well as peripheral equipment such as printers, scoreboards, etc, these not shown in the diagram.

Applied force is transduced in the load cell, to a voltage proportional to the applied force. This voltage signal output can be amplified and processed by conventional electrical instrumentation locally or externally to the pod 205.

An accelerometer, a device measuring acceleration in a particular direction, can also be used as sensor in the pod 205 to determine force, work and power.

Because an objective of the invention is to provide exercise data in most environments, flexibility is designed to accommodate many existing systems, exercises and exercise regimens. In some embodiments a wall track is not used, the device pods 205 are mounted to a free-standing pole, secure mounting structure or attached to a cable.

In embodiment where a stable attachment point is not accessible, a pod 205 containing two load cells and/or two strain gauges or transducers can be designed to be supported by the user. The pod 205 casing would look similar to the one with an opening at each end for coupling with exercise equipment and hands/grips. Stretching bands or inelastic cables, etc. could be inserted into both openings, and the user could pull them in any direction to achieve monitored exercise. Data could be transmitted wirelessly or via wires for processing. This embodiment could be expressed with the various monitor configurations detailed above and others.

In some embodiments, exercise signals are transmitted to a remote server so that a therapist, doctor, trainer, or other evaluator can observe the performance of the user in real-time on on-demand. A remote monitor may be a remote device such as a PDA/cell phone client.

Isometric Exercise

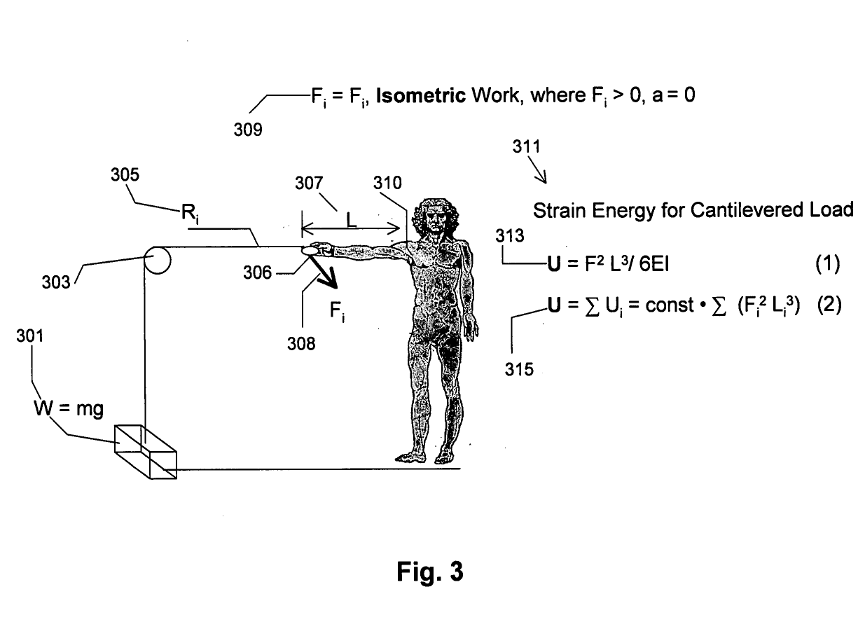

FIG. 3. illustrates a simple isometric exercise with accompanying force and strain energy model for data acquisition in accordance with an embodiment of the invention.

Work in the field of Physics is defined as a force vector displacement dot product, or in a simplified one dimensional case, force multiplied by the action resulting displacement. Where F 308 is greater than zero but the acceleration of the object of applied force is zero, work can be calculated in terms of strain energy 309. Hence physiological or muscle work can result without displacement, as in isometric exercise, and is modeled by strain energy methods. An arm pulling or pushing but not achieving any displacement can be modeled as a force vector acting on a cantilever beam of non-uniform cross section. As the force, F, is applied to the beam, the external work Sₑ is

Sₑ=P*deflection/2 (1)

where deflection is the total deflection at the hand where a concentrate force F 308 is applied. The strain energy is the bending and shear energy stored all along the cantilever, arm 307, from the force 308 to the anchor point, shoulder 310. The strain energy is predominantly in flexure bending stress, and is formulated to be S_{e-b}=F²L³/6EI. Where L is the length of the cantilever 307, I is the moment of inertia and E is Young's Modulus. Obviously I and E are not given physiological constants and the cross section is not uniform along the length of an appendage. The mechanics of fiber flexure of inanimate and animate tissue are different but the physics is similar and analogous, as both must transfer a point load F 308 and moment from that load to the cantilever base, or shoulder 310. Moreover, cantilever arm length can vary and is a variable that can be changed by bending the arm or changing the position from which a user pulls or pushes. Hence the strain energy models apply reasonable constants where useful to account for variables not changeable by users and measure or account for the variables that are and will be changeable by users, remaining faithful in processing the data to obtain valuable results for reporting. In this example, the isometric work may be S_{w}=constant*F²L³, where the constant can be formulated generically from averages or individually per user.

Hence an object 301 with an acting force f 308, here the object resistance is weight 301 over a pulley 303 typical of many exercise systems, is shown. The work U is given in the formulation 313 for a strain energy for cantilevered load 311. This is descretized for the strain energy shown in equation (2) 315.

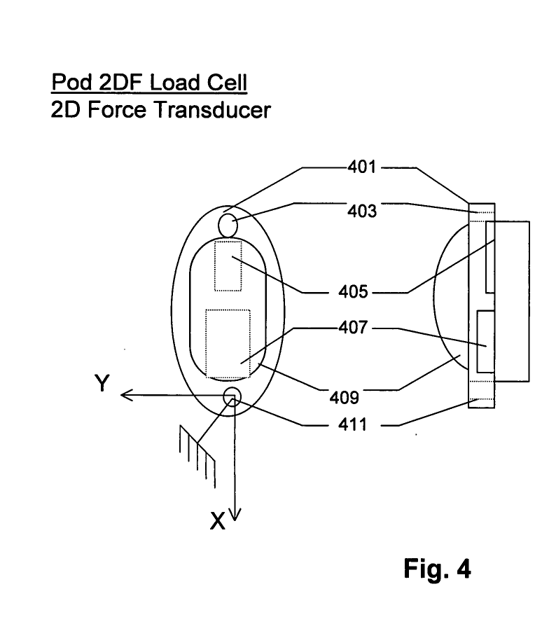

FIG. 4 shows front and side view for a load cell for 2D force vector in accordance with an exercise data system embodiment of the invention

A pod 401 casing 409 houses the load cell 405 and circuitry 407, providing a rigid coupling element with openings 403 411 for coupling devices, connecting a handle or gripping 411 and a resisting element or resistance transferring element on one and a rigid connection on other end. The two degree of measurement 2DF pods detect forces along two dimensions during an exercise and transmit raw data to the computer for processing. Each pod 401 contains a load cell 405 and circuitry 407 where conversion and processing can be done respectively, and signal outputs or data streams of the measured parameters can be transmitted for further processing, storage or display. In some embodiments, the circuitry is used to convert the data stream from the load cell 405 from analog into digital format. Pods 401 may optionally be communicatively coupled with “WiFi”, Blue Tooth, 802.11B, or similar means of wirelessly transmitting standards and formats. The pod 401 output force can be transmitted by rigid contact with a stretchable elastic or cable. The other pod end 411 can be rigidly anchored in the x-y orthogonal axis. A junction box further integrating load cell signals, instrumentation such as indicators, signal conditioners and the like, as well as peripheral equipment such as printers, scoreboards, etc, these not shown in the diagram. Where exercise equipment has adjustable heights for aligning the exercise movements, a 2 dimensional pod or load cell is sufficient to provide vector force and a calculatable displacement locus, based on elastic or cable characteristics.

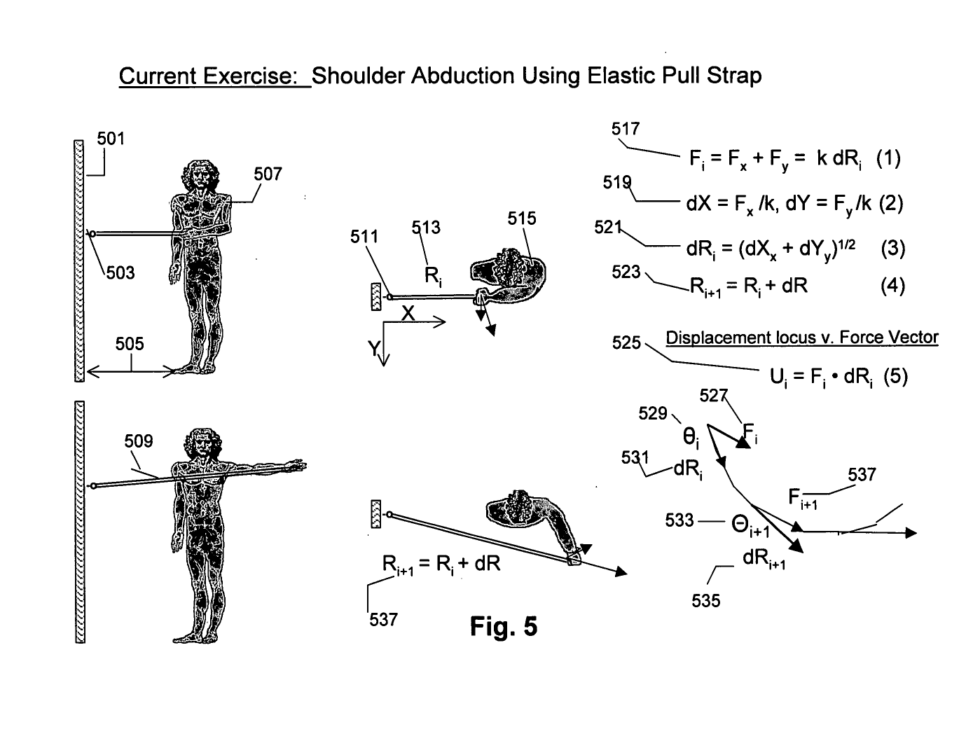

FIG. 5 shows front and top view for a shoulder abduction exercise with force vector and displacement for the purposes of determining force and work done on muscle from the elastic stretch bands resistance in accordance with an embodiment of the invention.

A typical exercise would affix a pod 503 to an anchor point 501 wherein the subject would be positioned a known distance 505 away from the anchor 501. The anchor point 501 position can be adjustable to a shoulder height such that the elevation component in the exercise does not change significantly. Thus only the X and Y dimensions 511 will require force and displacement measurements and the 2D-F transducer pod can suffice. Two pods offering only 1D-F each can also be set up orthogonal to each other or other coordinate systems can be used to provide the full 2D force and associated displacement measurements during the exercise so far as the calculations account for the measurements in the proper dimensionality scheme.

The Elastic Stretch

Where the elastic stiffness or spring constant is unknown, they may be calibrated by many methods, stretching the elastic 509 a known displacement 513 537, hanging or subjecting the elastic 509 to a known weight or force and noting the stretch length, etc will provide a spring constant in force/length units. Where the elastic material spring constant is non-linear, the non-linearity can be programmed in as a function of stretch length, thus allowing for the non-linear nature of some materials otherwise still suitable as exercise accoutrements.

The orthogonal force components, Fₓ+F_{y} sum to provide the instantaneous force Fᵢ 517. Accompanying the force is the displacement which can be calculated by components dX=Fₓ/k, dY=F_{y}/k, 519 or by total force k dRᵢ 517 using the geometrical parameters in spatial coordinates 521 dRᵢ=(dXₓ+dY_{y})^{1/2} and/or displacement vector 2D formulations 523 Rᵢ₊₁=Rᵢ+dR, where the start positions Rᵢ are known and the component displacements are calculated from the change in stress components 519.

The work Ui 525 is calculated from the force vector displacement dot product, Fi*dRi, component by component. This can be done incrementally by measuring and calculating Fi 527 and dRi 521 and multiplying their product by the projected angle θᵢ 529 between those vectors, repeating this for Fi+1 537 and dRᵢ₊₁ 535 and multiplying their product by the projected angle Θᵢ₊₁ 533 and so forth from the beginning to end of the pull exercise. The work and any point is faithfully calculated or projected force on displacement accounted.

Sensors, Systems and Monitors

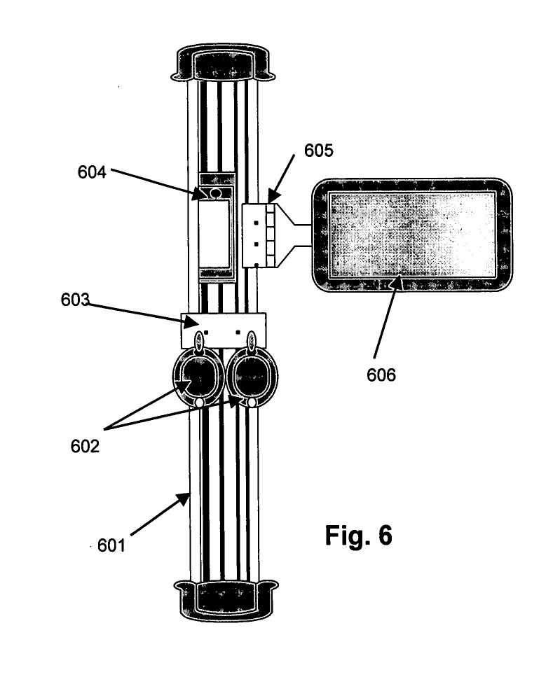

FIG. 6 shows an implemented exercise data system in accordance with an embodiment of the invention. A wall track 601 rigidly mounts the device to a wall, pole, or other secure object. Wall track has channels that can cover power cord(s), phone line(s), etc. for safety and aesthetics.

Pods 602 contain load cells and electronics. In some two-end pod elements, one end of the pod anchors to the wall track and the another end connects to the elastic cable or stretching band. Pods can be rigidly connected in any of one, two or three dimensions.

A U-bolt 603 or universal joint or ball-in-socket joint or similar system, couples a pod 602 to the wall track 601 and allows the pod 602 to be freely adjustable to a desired position on the wall track 601.

Sliding mechanisms 604 allow pods to be positioned at any height from the floor, to perform exercises on various targeted muscles of the body. Some embodiments may also contain electronics for various uses such as user height or position sensors.

Hinged and sliding attachment devices 605 support displays or monitors to be located at any desired height or angle for ease of viewing.

Housing 606 contains a monitor that is either integrated with a computer or that operates remotely. It may be wireless, depending upon the needs of the user. In this embodiment, there are specialized electronics in the monitor casing.

Some embodiments of the invention monitor resistance or tension forces generated during an exercise allowing the user three dimensional extension. In at least one embodiment, a wall track is coupled to one or more cable attachment points for elastic or inelastic exercise materials, load cells to continually measure resistance, a movable slider 605 that secures the attachment point(s) to the wall track 601 and allows the attachment points to move up or down to accommodate exercises with any limb in most any position, at least one pod 602 to house each load cell, circuitry that converts the load cell signal from analog to digital, a wireless transmitter to send the digitized signal to a computer, separate circuitry to further process the signal and convert it into a format that can be processed by a standard PC-type computer thirty times each second, and a monitor that can be adjusted so that it can be viewed at any angle. Various wireless and wired monitors 606 and monitor types may be used for exercises in which the user is not facing the wall-track.

In some embodiments, the pod 602 device is designed so that it can be mounted to a doorframe, table leg, bed frame, or other common sturdy attachment device commonly found in a home or office, or easily installed in a home or office. Other embodiments have a wall-track mounted system using a Tablet PC, wall-track mounted system using a PDA, touch-screen 606 mounted to the wall but not attached to the wall track, keyboard is mounted to the wall with touch-screen virtual keyboard, etc. Many such embodiments are possible. In some embodiments, the monitor is not attached to the wall track. It can be on a swing arm, on a cart, on a stand, or on any device that allows the user to position the monitor where he wants it to be: in front of him/her, to either side, behind the user, etc.

In some embodiments multiple monitors will be used. One can be mounted on the wall or wall track, and a second mounted on a cart. In still other embodiments, pods will not be attached to a wall track. They can be secured to a door frame, a door knob, a table leg, a chair leg, a hook fastened directly to a door, etc. Any stable attachment point may be appropriate.

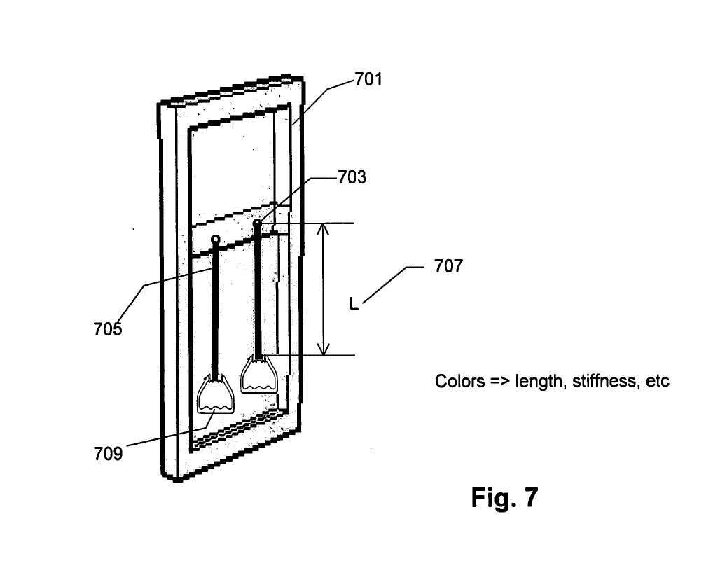

FIG. 7 shows two stretch bands and components of an isotonic exercise data system in accordance with an embodiment of the invention. A frame 701 is used to anchor pods 703 at one end and elastic stretch elements 705 from the other end. The elastic stretch elements measure a non-stretch length L 707 and are coupled with hand grips or handles 709. The spring coefficient of the elastic 705 can be known from a prior known and established color scheme which can be mapped to elastic element thickness, length, material properties, stiffness etc. These can be user selectable an easily attachable to the pods 705, to provide a variety of muscle regime quickly available and convenient exercise equipment. Where a pre-determined scheme is not used, a calibration of the kind mentioned can quickly establish the spring constants of the resistance elements used in the system.

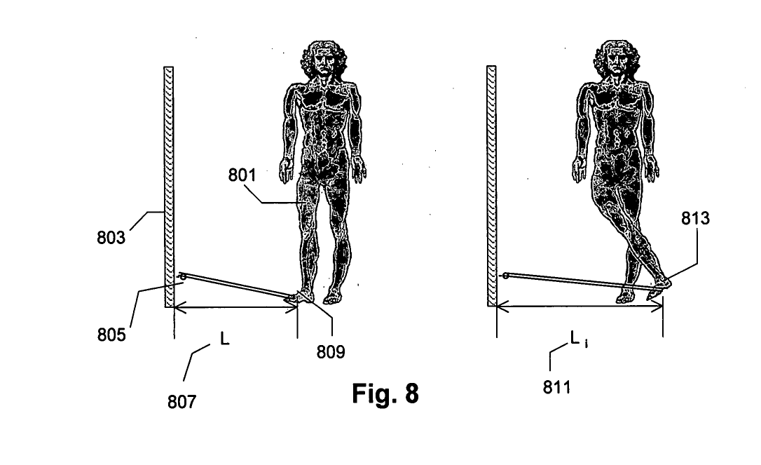

FIG. 8 shows a simple calibration and initialization for an exercise using an exercise data system in accordance with an embodiment of the invention. Muscle(s) 801 or groups identified or targeted for data acquisition and processing are selectively assigned a specific exercise. Case in point, the inside lower tie muscle(s). A connector is strapped to the subjects foot or ankle 809 a known distance L 807 from the pod 805 anchor 803 point. The exercise requires some muscle activity to move the resistance a displacement L 811. The pod registers the orthogonal forces in the plane of action, again the elevation is held constant, allowing the measured forces to be used to determine the affect, work and performance on the upper leg muscle(s).

It should be noted that the elevation adjustment can be made to target many muscles and muscle groups, by adjusting the elevation component, and position of the subject.

In addition to the spatial considerations of muscular activity, are the temporal aspects of muscle action. The work calculated for an exercise executed swiftly will by the conventional definition return the same number for the same exercise executed more slowly. This would be an incorrect formulation for the actual work, power, physiological work, power or strain energy. Thus the time over which the exercise spans must be factored into to calculate the physiological work involved in any particular exercise.

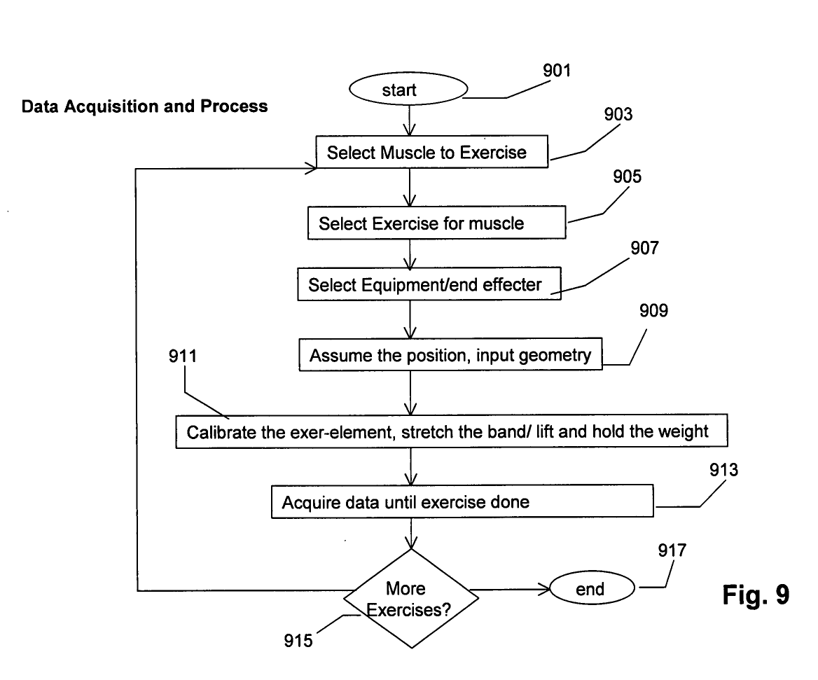

FIG. 9 shows a high level flow chart for exercise data acquisition and initialization process for an exercise data system in accordance with an embodiment of the invention.

The initialization process for an embodiment of the invention starts 901 with a prompt for the user to select muscle or muscle group to exercise 903. This may come from a pre-selected list or the users own making. This is followed by a selection of exercise for the selected muscle 905 and a selection of equipment/end effecter 907. The user will assume the position shown for the exercise selected, whereby the input geometry 909 can be inputted. This may include a calibration for the exercise-element such as a stretch the band/lift and hold the weight 911 type calibration to ascertain the stiffness of the resistance element. The exercise then proceeds with acquisition of data until exercise is complete to user satisfaction 913, at which point the user can option to select another exercise 915 and go to new selection 903 or end 917 the program.

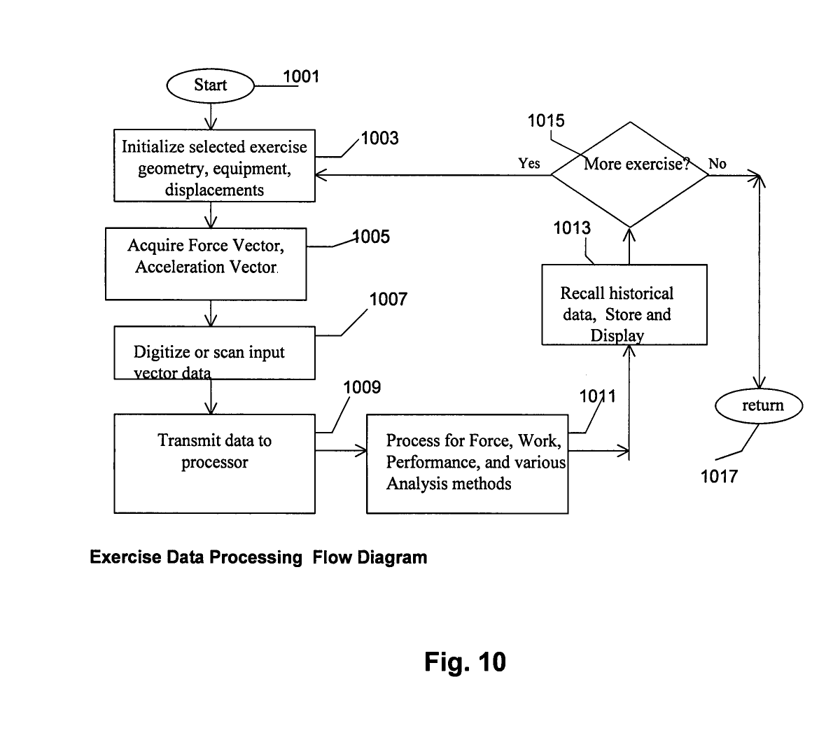

FIG. 10 shows a high level flow diagram for exercise data processing in accordance with an embodiment of the invention.

The processes begins 1001 to initialize the selected exercise geometry, equipment, displacements 1003 for the selected exercise or movement. This includes positioning the users stance away from the exercise point with the strap or cable extended to a known or determinable position. The acquisition element then acquire Force Vector, Acceleration Vector 1005 which are then scanned and digitized input vector data 1007. This data is transmit to the processor 1009 element where it is processed for physical Force, Work, Performance, various analytic methods and physiological resistance, work and performance 1011. Needed ancillary data, previous sets or historical data can be recalled, stored and/or displayed 1013 per exercise movement. Upon completion of the exercise the program will prompt user for other exercises 1015. An affirmative will loop back to selection of new exercise 1003 and a negative will complete the program with a return 1017.

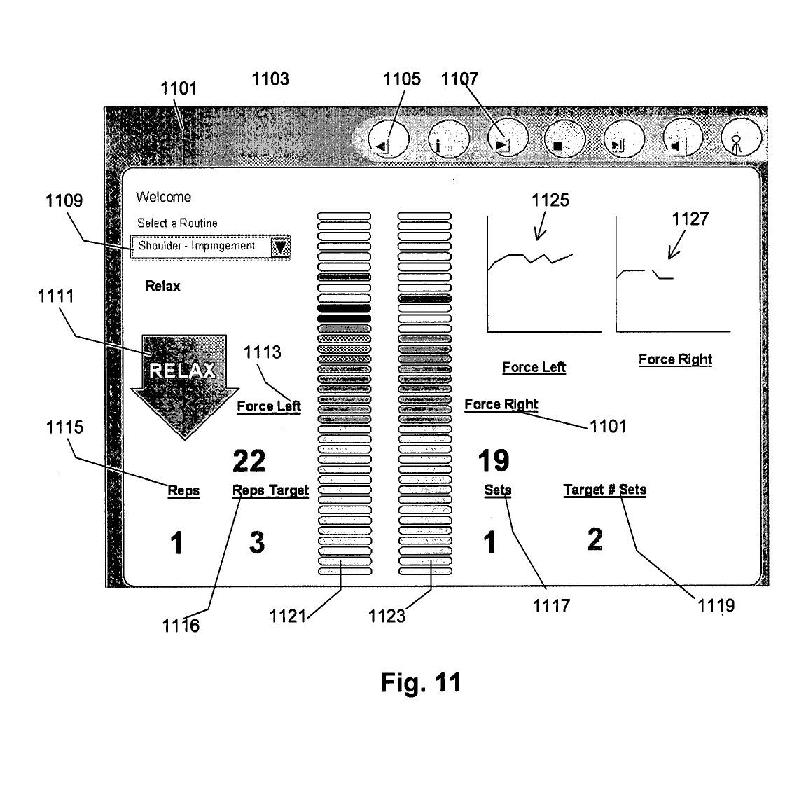

FIG. 11 illustrates an exemplar exercise data display in accordance with an embodiment of the invention. Buttons 1105 1107 allow the user to fast forward or reverse the data shown on the screen 1101, or load historic data from various stored previous exercise sets. Routine selection bar 1109 can is set by user for pre-programmed muscle and muscle group exercises. The Relax Arrow 1111 displays the relaxation periods, repetitions 1115 1119 and target number of repetitions are tracked and displayed as well as sets 1117 and target number of sets 1119. Left force 1113 and right force 1101 are displayed in bar graph format 1121 1123 respectively, adding color where appropriate to show good, bad, and warning messages. Graphs of the left 1127 and right 1129 applied forces for the exercise can also be display for historic, playback, comparison or real-time viewing purposes.

Processing Exercise Data

Captured exercise data can be used in many ways, to help the subject and/or evaluators understand underlying trends or problems that can ordinarily not be detected visually or by inspection. Mathematical and numerical processing of the data can yield insights from precursors, onsets or underlying muscle problems, not readily seeable, knowable or accurately understood. Smoothness, Normalized Resistance Differential Smoothness, Time Differential Smoothness, Fast-twitch muscle fiber processes, Maximum Force, Locus segmentation, Strain energy, Power workload, Percentage of workload, and Endurance Measurements and Scoring, etc form some embodiments of the invention. One method of revealing the unseen is via the smoothness characteristic using Resistance Locus Smoothness.

Exercise data smoothness character in individual repetitions or sets of an exercise highlights weaknesses at points along the measured parameter, resistance, through discontinuities in the curve locus. Weaknesses can be found in any part or range of motion, while force is being exerted against the stretching band or flexible material, and it points localized damage on a specific portion of a muscle. Smoothness can be studied in several ways.

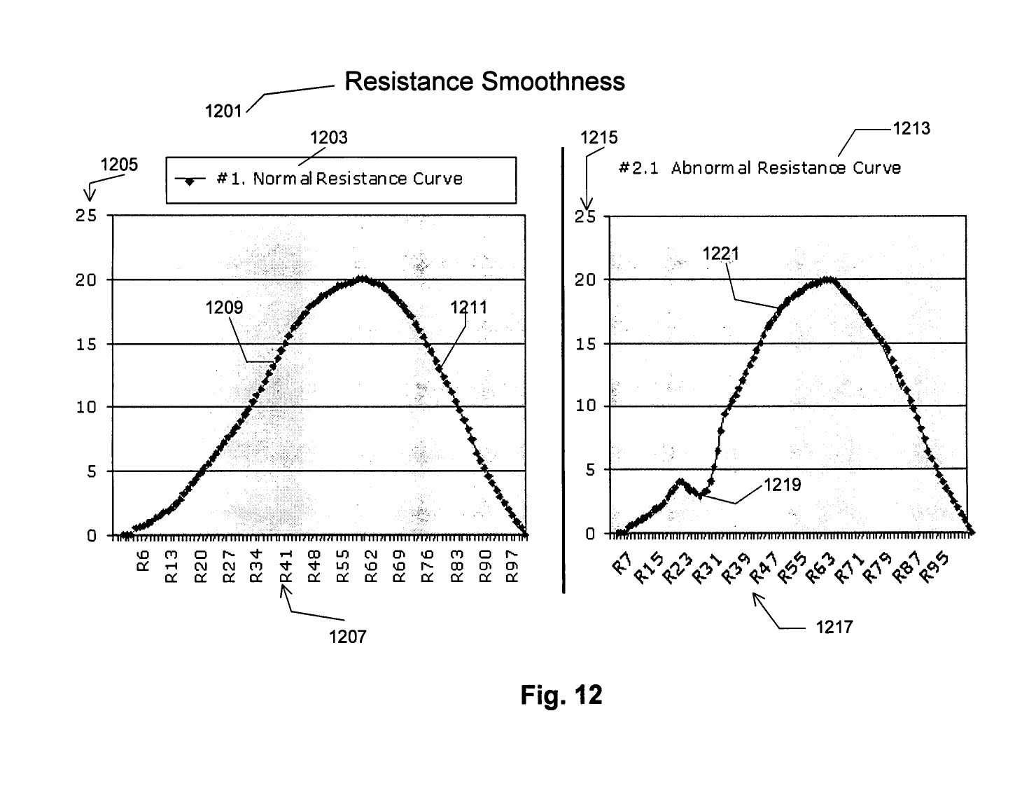

FIG. 12 illustrates exemplar exercise data graph 1203 for smoothness analysis 1201 in accordance with an embodiment of the invention. During a single repetition of an exercise with elastic or flexible materials, an aspect of the invention scans in and records the resistance force 1205 being exerted, scanning the action 1207 a multitude of times each second. On an embodiment of the invention, the resistance will begin at or near zero, increases on continuous monotonic path to a maximum, then decreases on a continuous path to zero or near-zero. The plot of these points Time 1207 vs. Resistance 1205 is called the Arc of Individual Repetitions with extension 1209 and contraction 1211 forming the increasing and decreasing parts of the graph respectively. The graph may be studied to reveal underlying phenomena not visually discernable or otherwise measurable.

“Smoothness” is an analysis of one or more locus or graphs of Individual Repetitions of an exercise and highlights anomalies at points along one or more discontinuities. Anomalies may be found at any section of an abnormal graph 1213 or set of graphs. They may indicate weakness or damage to a specific section of a muscle; the coordinates of the anomaly(s) can be used to locate the muscle damaged section. Smoothness can be studied in several ways:

Where a graph 1221 shows a break 1219 in smoothness in a range 1217 of motion. This may indicate a problem within the band of corresponding muscle that is fired when this point in the range of motion is reached. Graphs of a multitude of individual repetitions can be analyzed to determine if they show a pattern of similar breaks in smoothness to provide numerical data about its magnitude and point of discontinuity 1219, mapping that information to the location and muscle or joint of weakness from the range 1217.

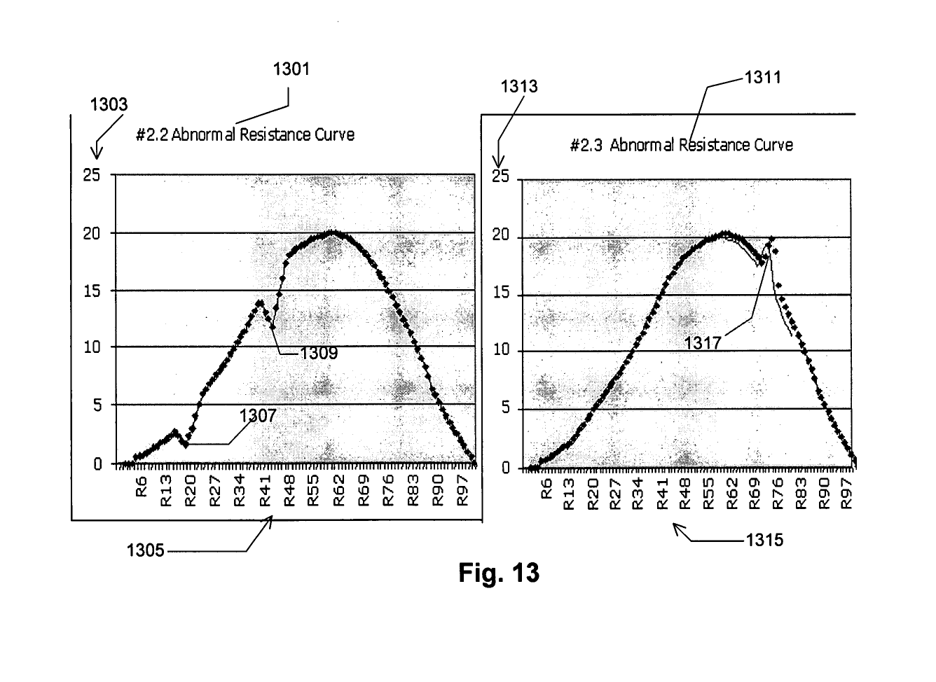

FIG. 13 illustrates exemplar exercise data identification of abnormalities from a smoothness analysis in accordance with an embodiment of the invention

An abnormality may show up in more than one discontinuities in the graphs or curves 1301 1313. The location on the graph is additional information. Discontinuities 1307 1309 1317 could indicate a tremor, a problem in a joint, or several other problems that a qualified doctor can diagnose. Graphs of a multitude of individual repetitions could be analyzed to determine if they show a pattern of similar breaks in smoothness.

The point of monotonic departure, the discontinuity 1307 1309 1317, sheds light on the nature of the anomaly and could help to isolate the nature of that problem, i.e. to distinguish between muscular inhibition caused by a problem with joint motion, guarding due to pain, buckling due to weakness, in the graphic dip 1307 1309 1317, or tremor due to fatigue, or movement dysfunction related to spasticity, graph showing a local transient discontinuity on an otherwise monotonic curve. Thus the shape of the discontinuity, one dip or a transient, and its position or range on the graph, at extension or contraction, all give measured response data. In theory, the precise muscle and position in muscle, joint or connector, can be found through a process of triangulation, where muscles near the area are exercised in different ways to narrow the zone of potential injury to the muscle and position in the injured muscle by concentrating the exercise set on a slightly different muscle until the anomalous discontinuity is exacerbated to its maxim measurable amount, thereby identifying the injury location more precisely. An example of this is shown in the difference position ranges 1305 1315 where the discontinuities 1307 1309 1317 arise. The discontinuities at 1307 and 1309 occur at extension of the exercise equipment, maximum contraction of the muscles, with 1307 occurring earlier in the extension indicating injury closer to the muscle connectors or joints, whereas the discontinuity at 1309 occurring later in the extension closer to larger force vector indicating an injury nearer to the center of the muscle. The discontinuity occurring on the relaxation portion of the curve 1317, may be an indication the muscle is fatigued and tiring, unable to sustain the contraction, with the injury causing the momentary involuntary relaxation.

A graph showing a flat range with a discrete step up, out of control, perhaps from an involuntary contraction. The cause could be neurological; it could from a poorly healed injury; and should be investigated further. Graphs of a multitude of individual repetitions can be analyzed to determine if they show a pattern of similar breaks in smoothness. Additional data adds information of a statistical nature which better localizes and characterizes the mapped injury.

Mathematical operations for Smoothness from the motion locus can be obtained by the following manner:
1. Capture or acquire a complete exercise repetition of N seconds with resistance being polled P times per second. Name this set of NP measures of resistance, Set S₁. Let X vary from 1 to NP.
2. Number the points in Set S₁ in the order in which they were polled: R₁, R₂, R₃, . . . R_{X} . . . R_{NP} so that each point R_{X} represents the force being exerted on elastic material at the time of measurement.
3. From Set S₁ create Set S₂, as follows: S₂={(R₂−R₁), (R₃−R₂), . . . (R_{X}−R_{X-1}) . . . (R_{NP}−R_{(NP-1)}}. S₂ is the set of differences between each adjacent pair of polled numbers. In general, these numbers will be positive as resistance increases, approach zero as resistance peaks, and then be negative as the pull on the elastic band is relaxed. Note that the individual positive values or Set₂ will not generally be the mirror image of the individual negative values.
4. Let R_{A} be the point at which maximum resistance was achieved. The values {(R₂−R₁), (R₃−R₂) . . . (R_{A}−R_{(A-1)}} form the Subset S_{2A}. The values {(R_{(A+1)}−R_{A}) . . . (R_{NP}−R_{(NP-1)}} form the subset S_{2B} The values in S_{2A} should be positive. Negative numbers in Set S_{2A} will indicate that the user's limb actually moved backwards for a short period. This is an immediate danger sign. It may indicate a weakness or defect in the section of the muscle(s) that control this portion of the range of motion; it may indicate a tremor or some other problem and bears further scrutiny.
5. The values in S_{2B} should be negative. Positive numbers in Set S_{2B} will indicate that the user's limb actually moved forwards for a short period. This is an immediate danger sign. It may indicate a weakness or defect in the section of the muscle(s) that control this portion of the range of motion; it may indicate a tremor or some other problem. This is a reportable event of a potential problem that requires further scrutiny.
6. It is anticipated that as a user approaches the maximum level of resistance, values in S₂ will be at or near zero. However, if there are one or more sections of Set S₂ in which the values are at or near zero followed by a section in which the values rise, this will indicate that the user may have struggled to get past this level of resistance. A pattern of a quickly changing locus throughout several repetitions will indicate to the evaluator that the muscle bears further scrutiny.
7. Similarly, if there are one or more section of Set S₂ in which the values decrease but do not approach zero followed by a section in which the values rise, this will indicate that the user may have struggled to get past this level of resistance. A pattern of these quick changes throughout several repetitions will indicate to the Evaluator that the muscle bears further scrutiny.

Normalized Resistance Differential Smoothness

Another mathematical operation on acquired exercise data embodiment of the invention is called the Normalized Resistance Differential Smoothness proceeds as following:
1. Capture a repetition of N seconds with resistance being polled P times per second. This creates a set of NP measures of resistance, called Set S₁. Let X vary from 1 to NP. Number the points in Set S₁ in the order in which they were polled: R₁, R₂, R₃, . . . so that each point R_{X} represents the force being exerted on elastic or flexible material at the time of measurement.
2. From Set S₁ create Set S₂, as follows: S₂={(R₂−R₁), (R₃−R₂), (R₄−R₃) . . . (R_{X}−R_{X-1}) . . . (R_{NP}−R_{(NP-1)})}. S₂ is the set of differences between each polled number; it should be uniformly positive and approach zero when resistance peaks.
3. Let Rₘᵢₙ=the resistance measured at the beginning of the repetition and let Rₘₐₓ=the resistance measured at the peak of the repetition. Therefore:
   a. Rₘₐₓ−Rₘᵢₙ=100D=the total change in resistance during a single repetition of an exercise. Also, D=1% of the total change in resistance.
   b. 100D/NP=A
   A=the average change in resistance between consecutive polling points during a single repetition of an exercise.
   c. Given any two consecutive points, R_{b} and R_{(b+1)},
   (R_{(b+1)}−R_{b})×100/(D)=the ratio between the measured difference between two consecutive points and A, expressed as a normalized percentage.
   d. Given any two points R_{b} and R_{(b+x)} where X is a positive integer less than NP: (R_{(b+x)}−R_{b})×100/(DX)=the ratio between the measured difference between two non-consecutive points and the average, expressed as a normalized percentage.
   e. If (R_{(b+x)}−R_{b})×100/(DX) produces a negative number equal to or larger than a pre-determined percentage of A, this indicates that the user lost muscle power during a section of the repetition when power should have steadily increased. The position of Point R_{(b+1)} or Point R_{(b+x)} will indicate precisely when and thus where in the muscle the user encountered the difficulty. This method has some distinct advantages over studying the raw data: it will indicate tremors that might not otherwise be obvious and it eliminates an occasional negative number that might be a meaningless artifact, such as a shifting in position of the elastic material.

A single negative number may have no significance but a multitude of negative numbers will indicate a problem to the user, therapist, or trainer. Negative numbers spaced closely together in time will indicate a weakness in a particular portion of a muscle; negative numbers that appear sporadically, in groups, may indicate a deeper problem that requires diagnosis.

The pre-set percentage of A will be determined for each individual user by the athletic trainer or therapist according to his/her training standards, thus allowing him/her to finely tailor each routine to the athlete or patient, programmed individual muscle scrutiny.

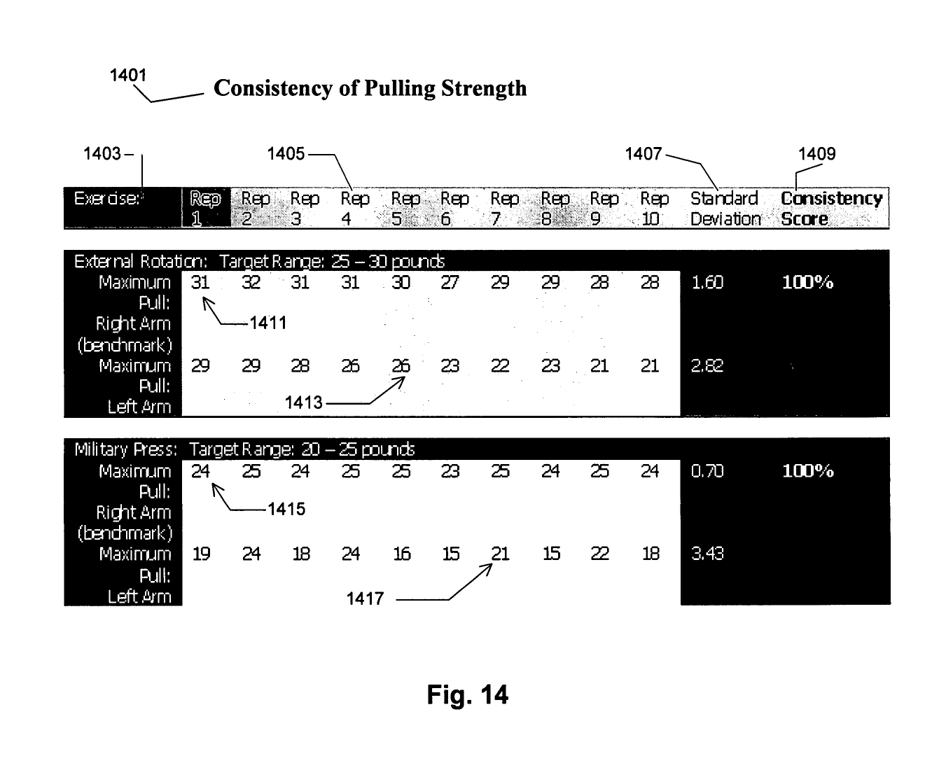

FIG. 14 shows a table of data used in exercise consistency analysis in accordance with an embodiment of the invention

Consistency of Pulling Strength 1401 compares the exercise 1403 1415 repetition set 1405 maximum pull of each rep 1411 1413 1415 1417 within a set 1405 to see if they fall within a normal tolerance. Consistency can be measured as a function of the Standard Deviation 1407 of the Maximum Forces 1411 1415, Force_{MAX}, exerted during each repetition 1411 1413 1415 1417 set.

A high percentage of inconsistent Maximum Forces 1411 1415 can act as a warning for the user. The user may have pain that varies depending on the precise motion s/he employs with a rep. S/he may have a weakness in a specific band of muscles for which s/he may or may not be able to compensate. The elastic exercise band being employed may simply be too hard to use properly. There are many possibilities that a therapist or trainer can investigate. Timely warning is key to quick rehabilitation.

Extremely consistent maximums imply that the exercise is too easy for the user and that he is performing mechanically. In this situation, the user should be instructed to increase the resistance. A report can help determine if a user's routine is at the right level of exercise—not too hard and not too easy, based on a consistency analysis score 1409.

Applications for the Consistency of Pulling Strength

An aspect of the invention compares the maximum force exerted during one or more repetitions of an exercise with an injured or affected limb to a benchmark of repetitions from the good limb. If no benchmark is available, standard scores can be used from a database or other source. For training two healthy limbs: First, compare scores from both limbs to ensure that they are similar. A wide discrepancy, to be determined by the Evaluator, indicates an unknown problem. Second, compare the score against the database or other sources to be sure that the user compares favorably.

In another embodiment of this analysis, only the last set of reps is analyzed as they show performance when a patient is tiring. The application of various consistency tests to different muscles and joints can reveal different weaknesses and onset or identification of physical problems.

Daily Trend of Endurance

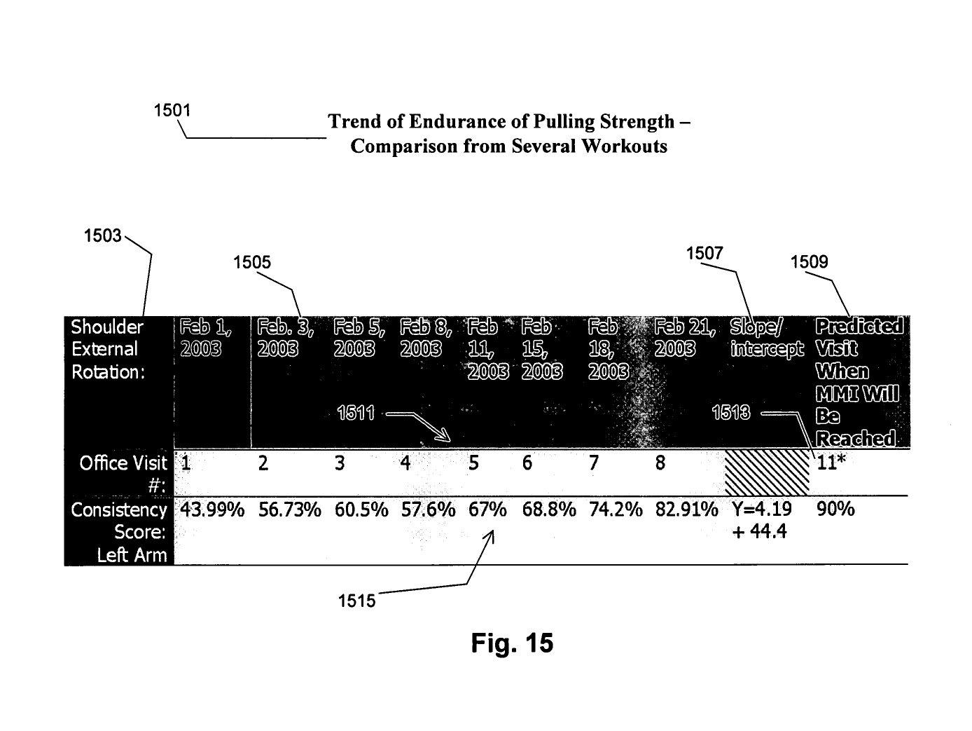

The FIG. 15 table of exemplar exercise report used in endurance trend analysis and shows the following steps are used for a selected exercise 1503 in a office visit 1511 sequence dated 1505 regimen. First, the maximum pounds lifted during each rep are used to compute maximum force for the set. Next, a benchmark is established by the healthy limb is set equal to 100%; the maximum from the benchmark is the divisor in taking the quotient and then percent of Score 1515. This will usually give a percentage less than 100%; when the Score 1515 reaches (i.e.) 90%, the patient will have been returned to MMI (Maximum Medical Improvement.) 1513. This method of using a regression of maximum scores in endurance exercise can be used to project when and the degree to which a patient is recovering.

An aspect of the invention computes the Trend of Consistency, which shows a user's progress: whether or not his workouts are growing more consistent over time. The program will compare the consistency scores to see if they are approaching 100%. This indicates that the patient is able to complete all three sets of each exercise, which demonstrates that his strength, endurance, and range of motion are all improving. For patients in already in therapy, therapists can use the Trend of Consistency to predict how many more office visits are required for the patient to reach MMI to some level acceptable to the doctor, therapist, provider.

Time Differential Smoothness

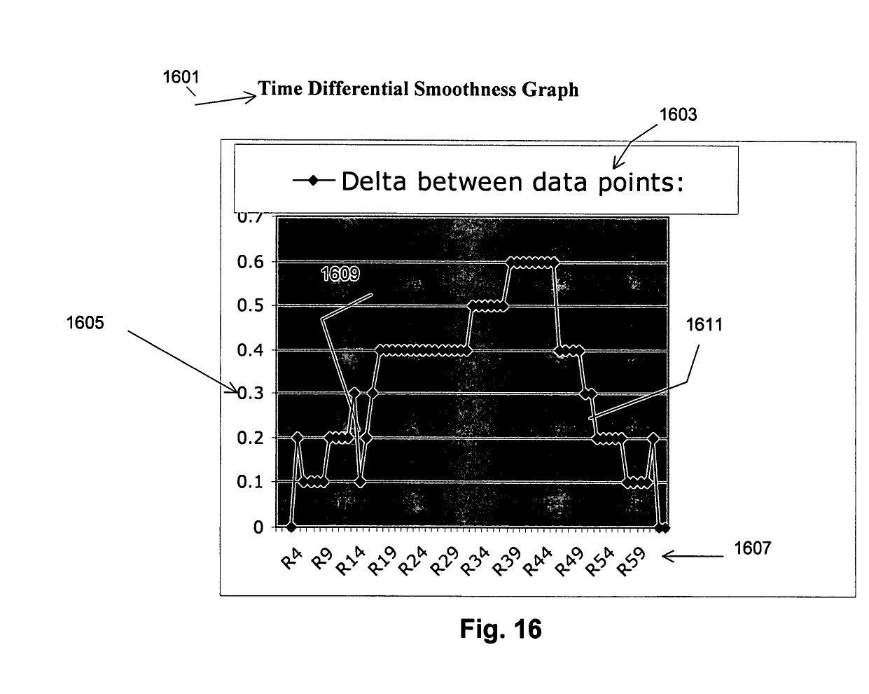

FIG. 16 illustrates exemplar exercise data graphs used in time differential smoothness analysis in accordance with an embodiment of the invention. Where the data may have looked smooth in a previous analysis, differential smoothness analysis plots the delta 1611 graph or exercise resistance difference between points 1605 as a function of data point 1607. This particular plot 1611 shows a discontinuity between in the R10-R15 range 1607. If this discontinuity 1609 occurs at approximately the same points in additional repetitions of the exercise, a symptom can be identified and a physical problem then diagnosed.

This numerical operation on acquired exercise data embodiment of the invention is called Time Differential Smoothness. Some objectives of Time Differential Smoothness are to determine if a particular section of muscle has been over-trained or under-trained and to non-invasively diagnose areas along a muscle that may be injured or impaired. Time differential studies as an embodiment of the invention can reveal the invisible, certain minor injuries can be diagnosed and therefore treated without surgery, MRIs, CT Scans, X-rays, etc. This is done through repeated time differential studies taken over a period of weeks or months which can show if an injury has healed, gotten worse, or remains unchanged. This data is acquired, processed and stored, for comparison and display progressively. The numerical processing is as follows:

1. The time-interval curve of the resistance phase of a single repetition {the graph of the changes in resistance from every pair in the set {(R₂−R₁) . . . (R_{A}−R_{(A-1)})} should be smooth and shaped like one-half of an inverted bell. The values should smoothly decrease and approach zero while the user pulls to the maximum force possible. Note that the values may not smoothly increase as the pull is relaxed during the second phase of the exercise; users may let a stretching band snap back under its own power.
2. If the time-interval curves of the resistance phase of several repetitions are not smooth, one section of values is consistently higher or lower than the surrounding values, then there may a problem with the muscle(s) that must be further diagnosed by a therapist or trainer.
3. Individual repetitions often have areas that are not smooth and this may be meaningless: a user may be interrupted, lose his/her grip, take a small step, etc. The Evaluator should look at all the repetitions to see if the problem recurs or if it was a one-time event.

Yet another process uniquely identifies the smoothness, or lack or smoothness of time-interval data graphs taken during an exercise. In at least one embodiment of the invention, a comparison and processing of time differential data proceed as follows:

1. Let T_{Min} be the time at which a repetition begins. Let T_{Max} be the time at which the repetition reaches maximum resistance. Then T_{Max}−T_{Min}=Tₛ=the number of seconds the user needed to go from minimum to maximum force.
2. Let R_{A}=the Average Resistance between consecutive polling points. Then:
3. Consider a repetition from a healthy, properly trained muscle. Given any two amounts of resistance (R_{B}) and (R_{(B+Y)}) then {100(R_{(B+Y)}−R_{B})} divided by Y should equal (R_{A}) plus or minus a pre-determined percentage. The pre-set percentage will be determined for each individual user by the athletic trainer or therapist according to his/her training standards and invention parameters, thus allowing him/her to finely tailor each routine to the athlete or patient. If there is a significant difference, the trainer etc. will be alerted that there is a problem.
4. In any set S as defined above, there may tens of thousands of pairs (R_{B}) and (R_{(B+Y)}) created during each repetition, making it difficult for the user, trainer, therapist, or other Evaluator, to evaluate each of them. Another embodiment of the invention is to automatically choose representative pairs (R_{B}) and (R_{(B+Y)}) that can be quickly evaluated by a user, etc. The chosen pairs, which will be displayed in several sets, will show samples of all meaningful time periods, so that the Evaluator can decide if there is a potential problem at any range. For example, data can be divided into set ranges:

Set #1 could consist of all pairs {R_{(B+1)}−(R_{B})} with the average of each result.

Set #2 could consist of all pairs {R_{(B+5)}−(R_{B})} with the average of each result.

Set #3 could consist of all pairs {R_{(B+10)}−(R_{B})} with the average of each result.

Set #4 Evaluator to select his own parameters for pairs (R_{B}) and (R_{(B+Y)}). For example:
a. Set #4 as consisting of all pairs {R_{(B+3)}−(R_{B})} with the average of each result.
b. Set #4 as consisting of all pairs {R_{(B+X)}−(R_{B})} with the average of each result, where X is any number from 1 to A where A is defined in 00124.4 above.

Unique results generated by embodiments of the invention to determine areas along bands of muscles that have been over-trained or under-trained, to determine areas that may be injured, weak, etc, and/or to adjust exercises based on this knowledge to improve workout routines.

Fast-Twitch Muscle Fiber Processes

To develop fast-twitch muscle fibers, a user will often perform an exercise as fast as is possible while reaching maximum resistance. In an embodiment of the invention, two separate sets of numbers, one generated during the time from rest to maximum resistance and the other generated during the time from maximum resistance to rest, are presented.

Raw times during the exertion phase and the relaxation phase must both be as short as is possible. These must be measured and stopwatches provide inadequate precision or time granularity to observe deviations from standpoint of feedback. An embodiment of the invention allows these numbers to be tracked over time, to show progress or decline to required granularity. Thus athletes develop their fast twitch fibers by tracking and analyzing how their muscles perform during exercise, and not only through the physical displacement of body and limb positions. In addition, presentation of movement data can describe how limbs accelerate from rest to maximum resistance, and how they then decelerate back to rest. By analyzing all the measurement points in Set S₁, an embodiment of the invention can precisely describe the maximum rate of acceleration in terms of the increase in pounds of resistance exerted.

Consider three subjects, A, B, and C, who perform identical exercises with the same limb. Assume they each take exactly 2 seconds to complete a single rep but have different acceleration analyses. The hypothetical results of the three analyses are:
i. A has an even rate of acceleration.
ii. B has uneven acceleration, with most of his acceleration coming in the early stage of the movement.
iii. C has uneven acceleration, with most of his acceleration coming in the late stage of the movement.

Presenting the processed results provides the evaluator the information necessary determine that subjects B and C do not have uniform rates of acceleration. This indicates the proper training to correct this imbalance.

Another embodiment of the invention provides accurate information on workload/sec and workload/rep, briefly summarized below as:
(1) Workload/rep=the total amount of work performed during a single repetition.
(2) Workload/rep=Σ(P₁ . . . P_{NP})/N
   where
   N=the number of seconds required to complete the repetition
   P=The number of times the data is polled per second.
(3) Workload/sec=the amount of work performed during a single second of a repetition. An embodiment of the invention can process this number for any portion of the repetition, starting at any point in time Tₓ.
(4a) workload/sec=Σ(P_{X} . . . P_{(x+P-1)})/P, where P=the number of polling points per second.
   or, to determine the workload per second over an interval of time that does not equal a second, from T_{X} to T_{Y}:
(4b) workload/sec={Σ(P_{X} . . . P_{Y})/(Y+1−X)}*P where P=the number of polling points per second.

Weights and Other Methods of Resistance

When using weights, in at least one embodiment, the following definitions apply:

### (A) Force=F, ma, −kdx, mg

### (B) Work=Force·Distance, Moment×Moment_Arm

### (C) Power=Force·Distance÷Time=Work÷Time

These standard definitions are modified for application to subjects exercising with stretchable or flexible material, as the vector force varies linearly throughout the movement whereas the vector force on a free weight varies by the acceleration vector applied and its position vector, not necessarily the weights, but at the user's physical application point. The work is the vector Force and Displacement dot product.

In some embodiments of the invention, physics definitions for Force Work and Power are analogous to a physical muscle Force, Work, and Power. Some definitions of intermediate variables are defined as:
N=the number of seconds required to complete a repetition
P=the number of times the data is polled per second.
X=any integer from 1 to NP, so that 1≦X≦NP
T_{X}=polling point X=Time elapsed at polling point X
R_{X}=the force being exerted on elastic or flexible material at time T_{X} of measurement.
D=the distance traveled from rest to maximum resistance and back to rest. Distance can be determined by direct measurement or inferred from a table that derives distance as a function of (A) force and (B) the length and thickness of stretchable or flexible material, or use of spring constant.
D_{Y-X}=the distance traveled during the interval T_{X} to T_{Y}. Note that Distance is always positive, regardless of direction of travel.
Force_{SB}=Average amount of work performed at any specified instant in time (T_{X}) during a single repetition, determined by taking the average of many readings per second of the resistance being applied to the material as it stretches then relaxes
Force_{SB}=The average force generated on a stretching band during one repetition of an exercise.
Force_{SB}=Σ(R₁ . . . R_{NP})÷NP

In an embodiment of the invention, Force_{SB} generates a unique value for exercise with stretching bands that is reasonably equivalent to the standard textbook definition of Force that is applied to exercise with free weights.

Force_{MAX} is the instantaneous maximum force generated during a single repetition of an exercise.

1. Work_{SB} is the total amount of work performed with a stretching band during a single repetition

Work_{SB}=Force_{SB}·D/2

Work_{SB}={Σ(R₁ . . . R_{NP})÷NP}·D/2

This method for calculating Work_{SB} generates a value for an exercise with stretching bands that are reasonably analogous to the Work that is applied to exercise with free weights.

2. Work_{SEG} is the amount of work performed in the distance between D_{X} and D_{Y}.

Work_{SEG}=Force_{SEG}*(D_{Y}−D_{X})/2={Σ(R_{X} . . . R_{(X+Y)})÷Y}*(D_{Y}−D_{X})/2

This method can be applied to many segments, overlapping and/or discrete, within a complete repetition to see if work is unexpectedly higher or lower than expected. Any anomaly that is consistently seen in many reps indicates a potential problem in a specific segment of the muscle(s) or the joint(s) involved. The location of the anomaly may indicate the location of the damaged tissues.

3. Power_{SB} is the power exerted a extending a stretch band.

$$\begin{matrix}{Power}_{SB} = {Force}_{SB} \cdot Distance / 2 \div Time \\ = {Work}_{SB} \div N\end{matrix}$$

$$\begin{matrix}{Power}_{SB} = \{ \sum ( {R}_{1} … {R}_{NP} ) \div NP \} ( \cdot D / 2 ) ( \div N ) \} \\ = \{ \sum ( {R}_{1} … {R}_{NP} ) \cdot D / 2 \} \div {N}^{2} P\end{matrix}$$

The formula for Power_{SB} generates a unique value for exercise with elastic materials that is reasonably analogous to the standard textbook definition of Power that is applied to exercise with free weights, but using acceleration force in stead of spring resistance force.

4. Power_{Y-X} computed during the entire relaxation phase will show if a user controlled his movement or allowed the stretching band or free weight to do the work.

Power_{Y-X}=Force_{SF}·(D_{Y}−D_{X})/2÷(T_{Y}−T_{X})

Power_{Y-X}={Σ(R₁ . . . R_{NP})÷NP}·(D_{Y}−D_{X})/2÷(T_{Y}−T_{X})

An embodiment of the invention to calculate physical exercise power can be applied to many segments, overlapping and/or discrete, within a complete repetition to see if Power_{y-x} is unexpectedly higher or lower than expected. Any anomaly that is consistently seen in many reps indicates a potential problem in a specific segment of the muscle(s) or the joint(s) involved. The location of the anomaly will indicate the location of the tissues that are not performing as expected.

The above methods have many practical applications as individual repetitions and data sets of repetitions; which can be compared between workouts on different days, to demonstrate improvement or decline, etc. For example, the relaxation phase of a repetition, where the user moves his/her limb from maximum resistance to rest, is as important as the exertion phase. Users tend to let the elastic snap back under its own force but should control the movement. This is quickly registered and appropriately assessed for force, work and power on any particular muscle targeted.

5. Textbook definitions of Work and Power fail to properly describe isometric exercises or exercises in which little movement is required as they are not demonstrative of physiological work, only physics defined work. Hence an embodiment of the invention provides the translation from meaningful measurements to meaningful calculations through the use of strain energy and time power.

Intuitively therefore, there should be a another equation that involves the variables force and time but not distance since there is no movement but where users exert power in strain energy, a non-moving work. Moreover, results require time measurements that are accurate to the tenth or hundredth of a second. These would need to be taken thousands of times each day, rendering stopwatches impractical. Hence, in an embodiment of the invention workload is defined and calculated as force per second, the amount of force exerted during one second. Using the following formula, Workload can be measured starting at any measurement point in the repetition.

Workload=Σ(R_{X} . . . R_{(X+P)})÷P

Workload_{SEG} is the force per segment of time or force exerted during a segment of time of any length during a repetition. Then, Workload_{SEG} can be measured starting at any polling point in the repetition. Assume that segment T begins at polling point and R_{T} and ends at polling point T+X, then workload is calculated by:

Workload_{SEG}=Σ(R_{T} . . . R_{(T+X)})÷X

6. Percentage of Workload exerted during a specific segment T of a repetition as compared to the workload for the entire repetition. Where that segment T begins at polling point R_{T} and ends at polling point T+X.

% Workload=100*Workload_{T}÷*Workload_{SB}

% Workload=100*{Σ(R_{T} . . . R_{(T+X)})÷X}÷{Σ(R₁ . . . R_{NP})÷NP}

% Workload=100*{Σ(R_{T} . . . R_{(T+X)})*NP}÷{Σ(R₁ . . . R_{NP})*X}

SDF

7. Power_{SEG} is the power exerted during any segment of time

$$\begin{matrix}= {Force}_{SB} * ( {D}_{Y} - {D}_{X} ) \div ( {T}_{Y} - {T}_{X} ) \\ = \{ \sum ( {R}_{1} … {R}_{NP} ) \div NP \} \cdot ( {D}_{Y} - {D}_{X} ) \div ( {T}_{Y} - {T}_{X} ) \\ = \{ \sum ( {R}_{1} … {R}_{NP} ) * ( {D}_{Y} - {D}_{X} ) \} \div \{ NP * ( {T}_{Y} - {T}_{X} ) \}\end{matrix}$$

8. The Maximum Forces produced during one or more repetitions of one or more exercises. For example: Force_{MAX} equal the maximum amount of force exerted on a stretching band during a single repetition of an exercise. Then
ΣForce_{MAX} equals the sum of a series of repetitions. It can be the sum of a single set or the sum of several sets of reps.
ΣForce_{MAX} divided by the total number of reps is the average maximum force exerted during one or more sets of repetitions

A trend line of the Force_{MAX} taken from a series of repetitions can indicate if an exercise is too hard or too easy for the user. The average of the maximum forces taken for each day's exercises by averaging some or all of the values, the averages from several workout sessions can be analyzed to show if the User is improving over time. Many other analyses can be done for the Force_{MAX} values and the ΣForce_{MAX} values and only a few basic applications are discussed here as a fundamental cycle of operation.

An embodiment of the invention will analyze repetitions performed by a user in several ways, including but not limited to:
a. Analyzing each measurement within each repetition of an exercise. Measurements can be analyzed for individual repetitions, sets of repetitions, or workouts on different days.
b. Analyzing the Maximum Force exerted during each repetition of an exercise. Maximum force can be analyzed for individual repetitions, sets of repetitions, or workouts on different days.
c. Analyzing the time required to get from any point during a repetition to a second point during the same repetition, such as from start to Maximum force. Time can be analyzed for individual repetitions, sets of repetitions, or workouts on different days.
d. Analyzing data with any of the above techniques and/or other techniques or combinations of techniques to predict trends and future performance.
e. Analyzing data with any of the above techniques and/or other techniques or combinations of techniques to reveal or to discover indications of problems graphically from time trends or special discontinuities indicating such as general muscle weakness, muscle weakness in a specific area only, range of motion problems and limitations, tremors, unusual difference(s) in abilities between limbs, fatigue problems, endurance problems, consistency problems, unusual differences between a specific user and the general population or any subset of the general population from database or from other sources.

Endurance Measurements

Endurance measures how quickly a user tires during an exercise. Endurance should be roughly the same for both limbs if they are both normal. A measurable threshold differential will indicate a symptom.

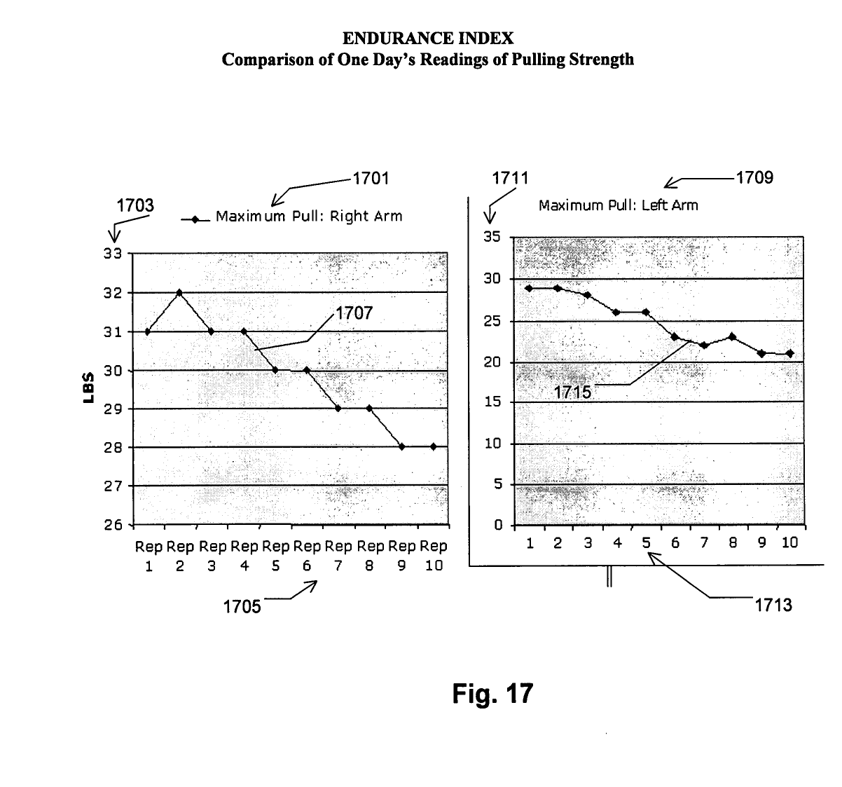

FIG. 17 illustrates exemplar exercise data graphs used in comparison and trend analysis in accordance with an embodiment of the invention

In another embodiment of the invention is the Daily Endurance Score for an exercise, measuring the Maximum Pull 1701 1709 in each repetition during one or more sets of repetitions 1705 1713 respectively and comparing it to benchmark data. In one embodiment, every repetition in every set is examined. In another embodiment, only the last set of repetitions is examined as these final repetitions show performance when a patient is tiring. In other embodiments, each set of repetitions could be studied separately. In still other embodiments, the final repetition of each set (or the final two reps, final three reps, etc.) could be studied and compared as they come just before a rest period.

Although the graphs 1707 1715 do not show it well because of the different ordinate scales 1703 1711 respectively, the slope of the maximums of the left arm 1715 is significantly more negative than that of the right 1707, the right arm is stronger, the left arm tires more easily.

When treating an injured or otherwise impaired limb, one method takes measurements from both limbs and uses the results from the healthy limb as benchmarks. Where this is not possible, benchmarks from a general database created from many users or from other sources can be used.

Computing the Daily Endurance Score

In one embodiment of a formula to compute The Daily Endurance Score, the maximum force pulled during each repetition is plotted for each set of Maximum Pulls as a straight line, linear regression analysis may be used. The slopes for both the affected limb and the unaffected limb are compared, the unaffected limb provides the benchmark slope, the benchmark slope by the slope from the treated limb and multiplied by 100 to produce the score. Scores will be lower than 100, unless the treated limb has more endurance than the healthy limb.

The benchmark slope is set at +100%. In therapeutic situations, when the Daily Endurance Score reaches 90%, or some other pre-established limit, then the treated limb has reached Maximum Medical Improvement (MMI) in terms of endurance. Note that 90% is an arbitrary figure and will vary from patient to patient. MMI is decided by the therapist or doctor.

In some training situations, if scores from two healthy limbs are being compared, a trainer might want to ensure that both limbs remain closely together in terms of strength and endurance. The trainer may choose any range (i.e. 95%) and work with the athlete to ensure that both limbs remain close together by monitoring the Daily Endurance Score. If the score slips below the set limit (i.e.) 95%, the trainer can work with the patients weaker limb to increase its strength and endurance. Different trainers may have different minimum ranges for the Daily Endurance Score, depending on the needs and abilities of the athletes.

In other embodiments, more sophisticated mathematical techniques replace linear regression analysis to provide deeper insight into the functioning and for improvement of a healthy or unhealthy limb through various diverse rehabilitation techniques.

Although the detailed description above contains specificities, these should not be construed as limitations on the scope of the invention, but rather as an exemplification of one preferred embodiment thereof. Other variations are possible. For example, the size of all of the components can vary, the shapes of the individual components can vary, the materials used for any component members may vary, the position and type of locking device can vary and the type of pivot mechanism is variable and the blade edge configurations are variable.

Therefore, while the invention has been described with respect to a limited number of embodiments, those skilled in the art, having benefit of this invention, will appreciate that other embodiments can be devised which do not depart from the scope of the invention as disclosed herein. Accordingly, the scope of the invention should be limited only by the attached claims. Other aspects of the invention will be apparent from the following description and the appended claims.

What is claimed is:

1. A system for acquiring, processing and reporting personal exercise data comprising:
transducer element registering an applied vector force from at least one muscle or muscle group acting on physical exercise equipment,
a sensing device converting vector force to electronic signal,
a data receiving element receiving the sensed signal;
an application program comprising a computing device processing the input from data receiving element, and
an application program processing vector force and data received from personal exercise,
whereby applied vector force on an exercise equipment element sends force vector and displacement vector data for determining resistive force done by a muscle or muscle group.

2. A system as in claim 1 further comprising determining work or power done by a muscle or muscle group.

3. A system as in claim 1 further comprising an personal exercise element from a set of exercise elements consisting essentially from elastic cable, stretching band, weight bearing cable, flexure member, spring, bar, rigid structure, non-stretching cloth, composites, elastics, latex, hypoallergenic elastic, flat sheets, tubes, slates, pipes and fiber.

4. A system as in claim 2 further comprising stretching bands of different colors representative of different thickness, different cross section or different stiffness of material.

5. A system as in claim 1 further comprising sensing vector force concurrent with processing associated displacement of personal exercise element end effecter.

6. A system as in claim 4 further comprising sensing vector force concurrent with processing associated displacement of personal exercise element end effecter from accelerometer vector data.

7. A system as in claim 1 further comprising resistance applied elastic or inelastic material personal exercise elements from the set of materials comprised essentially of man-made natural fiber, rope, webbing, steel cables, and composite wherein at least one sensor can sense force during user contraction over the user achievable range of motion.

8. A system as in claim 7 further comprising a material stiffness calibration of the exercise element within the user applied range, to determine stiffness constant over the user applied range.

9. A system as in claim 1 further comprising a geometric calibration of the exercise element initial configuration to determine force vector or moment arm length from applied vector force

10. A system as in claim 1 further comprising a 3D accelerometer coupled to the application program for acceleration vector data.

11. A system as in claim 10 further comprising determining the exercise machine weight load by sensing for vector force aligned with and opposed to the weight load while the weight is suspended and the acceleration vector sensed is negligible in the aligned vector force direction.

12. A system as in claim 1 further performing numerical integration on the accelerometer vector data to obtain force vector associated displacement locus.

13. A system as in claim 1 further performing a mathematical numerical dot product vector force data and the associated exercise displacement locus over the duration of the exercise to determine work performed by the users particular muscle or muscle group.

14. A system as in claim 1 further determining the isometric exercise strain energy work performed in an exercise by obtaining the product of the square of the force vector and the cube of the moment arm acting on that force vector with a constant of proportionality multiplier related to the cross section and cross section variability of the appendage acting as the moment arm.

15. A system as in claim 1 further determining the power exerted by user muscle or muscle in an exercise by a numerical technique of time integration over an applied force vector without associated displacement vector per unit of time.

16. A system as in claim 1 further determining the strain work exerted by user muscle or muscle in an exercise by a numerical technique of time integration over an applied force vector without associated displacement vector

17. System for acquiring processing and reporting generic, isometric, and or isotonic personal exercise data for selected muscles or muscle groups of a user, comprising:
an input device having at least a one dimensional force transducer;
a force applicator coupled to the force transducer wherein the force transducer registers the vector force applied to the applicator and generates a force signal representative of the vector force applied to the transducer by the user;
interface electronics for converting the force signals to receiver compatible output signals;
input-output components to receive signal from device interface electronics;
software instructions stored in memory for processing output force signal, under control of at least one processor communicatively coupled to interface electronics, instructions comprising:
establishing communication link from input interface electronics to processor,
establishing communication link from processor to an exchange server application or personal computing device configured to register a user;
transmitting the requested information to a display or reporting device, and
displaying or reporting the exercise data,
whereby a communicatively linked user can access and manage exercise information resident on networked servers spanning multiple organizations, health providers, health insurers and entities involved in the storage or processing of at least one individuals medical information.

18. A system for acquiring processing and reporting generic, isometric, or isotonic personal exercise data as in claim 17 further comprising
remotely accessing a user account from a server application;
obtaining a users login authority;
reporting exercise information;
comparing and processing with user information accessible by the server application

19. A system for acquiring processing and reporting personal exercise data on selective muscles as in claim 17 further comprising manipulating stored exercise information using a wireless device.

20. A system for acquiring processing and reporting personal exercise data on selective muscles as in claim 17 further comprising setting account preferences in a secured communication link.

21. A system for acquiring processing and reporting personal exercise data on selective muscles as in claim 17 further comprising setting account preferences in a secured communication link between wireless device and medical information system servers.

22. A system for acquiring processing and reporting personal exercise data on selective muscles as in claim 17 further comprising setting account preferences in a secured communication link between wireless device and medical information system servers.

23. A system for acquiring processing and reporting personal exercise data on selective muscles as in claim 17 further comprising an application with a user interface for navigating and displaying previous exercise data in grids, text or graphical formats.

24. A system for acquiring processing and reporting personal exercise data on selective muscles as in claim 17 further comprising off-line communication with medical professionals regarding users medical claims.

25. A method for processing and reporting personal exercise data comprising the steps of:
transducing applied vector force from at least one muscle or muscle group acting on physical exercise equipment,
converting vector force to electronic signal,
receiving the electronic signal in a computing device;
initializing and transmitting user position and exercise device position to the computing device;
transmitting exercise position displacement vector from position sensors to computing device;
processing received force vector signal and position vector to data in an application program;
storing vector force data and corresponding displacement vector received from personal exercise,
whereby stored force and associated displacement vector data from exercise applied to equipment element is used for determining resistive force applied by a muscle or muscle group.

26. The method for processing and reporting personal exercise data as in claim 25 further comprising the steps of determining the physics defined work by calculating the dot product between the force vector and the associated displacement vector.

27. The method for processing and reporting personal exercise data as in claim 26 further comprising the steps of:
accepting user selected exercise and equipment,
initializing the selected exercise geometry for the particular exercise equipment, scanning and acquiring displacement vectors and force vectors for the selected exercise motion,
transmitting data to the processor element, and
processing data for calculating physical resistance, work, and performance and
determining analogous physiological resistance, work and performance.

28. The method for processing and reporting personal exercise data as in claim 27 further comprising the steps of accessing previous sets or historical stored data for comparison, contrasting and display.

29. The method for processing and reporting personal exercise data as in claim 25 further comprising the steps of processing force and displacement vector data for numerical smoothness by plotting force vector resistance as functions of displacement, revealing uncharacteristic non-monotonic discontinuities in an otherwise monotonically increasing or decreasing graph, thereby allowing the mapping from point of graph discontinuity to displacement position and muscle physical symptom.

30. The method for processing and reporting personal exercise data as in claim 25 further comprising the steps of processing force and displacement vector data for numerical consistency of pulling strength by comparing the exercise maximum resistance within a repetition set for values within a preset tolerance level.

31. The method for processing and reporting personal exercise data as in claim 25 further comprising the steps of processing force and displacement vector data for numerical endurance trend analysis using a time history of a selected repeated exercise and stored selected exercise values, graphing the values as function of separate trials over a period of time sequentially dated regimen, numerically illustrating a particular muscle or muscle groups endurance strength over time for a selected exercise.

32. The method for processing and reporting personal exercise data as in claim 25 further comprising the steps of processing force and displacement vector data for numerical time differential smoothness, by graphing the mathematical derivative for the selected exercise resistance data graph, consistency graph or trend plot, highlighting departures from monotonic curves as potential symptoms of physical dysfunction, mapping non-uniform curve points to particular muscle.

33. The method for processing and reporting personal exercise data as in claim 25 further comprising the steps of numerically processing force and displacement vector data, comparing selected exercise data graphs of a known healthy limb with a suspect limb for consistency and trend over a period of time.

34. The method for processing and reporting personal exercise data as in claim 25 further comprising the steps of processing initialization geometry, force and displacement vector data for numerically calculating strain energy as modeled by a cantilever of non-uniform cross section about a fixed point.

35. The method for processing and reporting personal exercise data as in claim 25 further comprising the steps of processing force and displacement vector data for numerical trend of endurance measurements for a given period and selected limb and exercise.

36. The method for processing and reporting personal exercise data as in claim 25 further comprising the steps of processing force and displacement vector data for numerical consistency measurements, comparing measured values from a set of previous result sets, opposite limb measured results or other statistically obtainable basis data.

37. The method for processing and reporting personal exercise data as in claim 25 further comprising the steps of processing other physiological data from vital sign measurements corresponding to or concurrent with exercise
